(19) United States
(12) Patent Application Publication
(10) Pub. No.: US 2005/0153887 A1
(43) Pub. Date: Jul. 14, 2005

(54) Compositions for inducing cell growth and differentiation and methods of using same

(75) Inventors: Wange Lu, Pasadena, CA (US); David Baltimore, Pasadena, CA (US)

Correspondence Address: DLA PIPER RUDNICK GRAY CARY US, LLP, 4365 EXECUTIVE DRIVE, SUITE 1100, SAN DIEGO, CA, 92121-2133, US

(73) Assignee: California Institute of Technology, Pasadena, CA (US)

(21) Appl. No.: 10/972,073

(22) Filed: Oct. 22, 2004

Related U.S. Application Data
(60) Provisional application No. 60/561,324, filed on Apr. 12, 2004.
(60) Provisional application No. 60/514,491, filed on Oct. 24, 2003.

(51) Int. Cl. A61K 38/17; G01N 33/53; G01N 33/567

(52) U.S. Cl. 514/12; 435/7.2

(57) ABSTRACT

Compositions, including, for example, soluble Ryk polypeptides and Wnt polypeptides, that induce cell growth and/or differentiation, including, for example, neurite outgrowth and hematopoietic cell proliferation and differentiation, are provided. Methods of using such compositions also are provided, including, for example, methods of using such compositions to induce neurite outgrowth (e.g., in a subject having a neuronal disorder). In addition, methods to identify agents that alter Ryk mediated signal transduction in a cell are provided.

### CROSS REFERENCE TO RELATED APPLICATION(S)

This application claims the benefit of priority under 35 U.S.C. §119(e) of U.S. Ser. No. 60/514,491, filed Oct. 24, 2003, and U.S. Ser. No. 60/561,324, filed Apr. 12, 2004, the entire content of each of which is incorporated herein by reference.

### GRANT INFORMATION

This invention was made with government support under Grant No. 5R01 CA 51462-15 awarded by the National Institutes of Health. The United States government has certain rights in this invention.

### BACKGROUND OF THE INVENTION

1. Field of the Invention

The invention relates generally to cell biology and molecular pathology, and more specifically to the identification of role of the Ryk cellular receptor in Wnt mediated signal transduction; to methods of identifying agents that alter the interaction of Ryk with Wnt mediated signal transduction pathway proteins, including, for example, Wnt, Frizzled, and Disheveled; and to methods of using a soluble Ryk polypeptide and/or Wnt polypeptide to modulate cell growth and proliferation, including, for example, neurite outgrowth.

2. Background Information

The human nervous system is perhaps the most complex structure in the body. Unfortunately, many disorders of the nervous system exist, presenting serious and challenging health problems. Neuronal disorders are diverse, chronic, challenging to treat, and often disabling. They can be caused by many different factors, including inherited genetic abnormalities, problems in the immune system, injury to the brain or nervous system, or diabetes.

Among the many neuronal diseases, neurodegenerative diseases present a particularly serious health problem, with approximately 5-6 million Americans currently afflicted with chronic or acute neurodegenerative diseases. Neurodegenerative diseases are a heterogeneous group of disorders including, for example, Parkinson's disease, Alzheimer's disease, amyotrophic lateral sclerosis, Huntington's disease, and spinocerebellar ataxias.

Parkinson's disease and Alzheimer's disease are two of the more well recognized neurodegenerative diseases. The neurodegenerative process in these diseases is characterized by extensive loss of selected neuronal cell populations accompanied by synaptic injury and astrogliosis. Parkinson's disease is a neurological disorder further characterized by the loss of neurons, resulting in coarse tremors, muscular rigidity, and emotional instability. Alzheimer's disease results in loss of memory, cognitive impairment, and eventual dementia. Typical pathological characteristics include amyloid-containing neuritic plaques and neurofibrillary tangles. There are currently no cures for these neurodegenerative diseases, and they are progressively debilitating and ultimately fatal.

Progress is being made in understanding the mechanisms responsible for the dysfunction and death of neurons in many neurodegenerative disorders, but, unfortunately, very few treatments have been advanced. Current treatments are particularly limited in their ability to stimulate repair or regeneration of neurons. Mature neurons generally do not divide or reproduce and are limited in their ability to regenerate, with a full quota of neurons existing at birth or shortly thereafter. As such, neuronal damage sustained during injury or disease is often permanent. Therapeutic agents that have been developed to retard loss of neuronal activity and survival have been largely ineffective. Many therapeutic agents are not only limited in their effectiveness, but also have toxic side effects that further limit their usefulness.

Thus, a need exists for methods and compositions for treating neuronal diseases. In particular, there is a need for treatment compositions and methods capable of stimulating neuronal growth and regeneration.

### SUMMARY OF THE INVENTION

The present invention is based on the discovery that the membrane bound mammalian Ryk polypeptide acts is involved in the Wnt signal transduction pathway. As disclosed herein, Ryk specifically binds to Wnt polypeptides through its extracellular Wnt inhibitory factor (WIF) domain, and to the cysteine-rich domain of the membrane bound Wnt binding protein Frizzled. As such, Ryk acts as a co-receptor for Wnt binding. In addition to establishing Ryk as a functional receptor for Wnt, the present results demonstrate that Ryk binds Dishevelled, which is an intracellular scaffold protein involved in Wnt signaling, thereby providing a link between Wnt and Dishevelled. The present invention is further based on the discovery that Ryk is required for transduction of Wnt signaling, including Wnt-mediated T cell-specific factor (TCF) activation and Wnt-mediated neurite outgrowth.

The present invention relates to a method of inducing neurite outgrowth. In one embodiment, the method of inducing neurite outgrowth is performed by contacting a neuronal cell or a neuronal precursor cell with a Wnt polypeptide. The Wnt polypeptide can be any Wnt polypeptide that induces neurite outgrowth, including, for example, Wnt 3a (e.g., SEQ ID NO:2, SEQ ID NO:4), Wnt1 (e.g., SEQ ID NO:6, SEQ ID NO:8), and Wnt4 (e.g., SEQ ID NO:10, SEQ ID NO:12), or a peptide functional fragment of a Wnt polypeptide that selectively bind Ryk and Frizzled.

As disclosed herein, Wnt signal transduction and Wnt induced neurite outgrowth involves Ryk, and soluble Ryk enhances the neurite outgrowth inducing effects of Wnt. As such, the method of inducing neurite outgrowth can further include contacting the cell with a soluble Ryk polypeptide. The soluble Ryk polypeptide can comprise any portion of a Ryk polypeptide that is soluble and enhances Wnt induced neurite outgrowth, including, for example, a polypeptide comprising the extracellular domain of a Ryk polypeptide (e.g., about amino acid residues 42-224 as set forth in SEQ ID NO:14, or about amino acid residues 36-211 as set forth in SEQ ID NO: 16).

Neuronal cells or neuronal precursor cells that can be contacted with Wnt, alone or in combination with a soluble Ryk, such that neurite outgrowth is induced, and cells can be any neuronal cells or neuronal precursor cells that are responsive to Wnt and include Ryk, or a Ryk homolog (e.g., Linnotte or Lin-18) in the Wnt mediated signal transduction pathway. As such, the neuronal cells or neuronal precursor cells can be vertebrate cells, including, for example, mammalian cells (e.g., human cells). Further, the cells can be cells of an established cell culture, or can be primary neuronal or neuronal precursor cells obtained from a subject, including a normal, healthy subject or a subject having a neuronal disorder such as a neurodegenerative disease (e.g., Alzheimer's disease, Parkinson's disease) or a traumatic nerve injury. According to the present methods, the cells can be contact with a Wnt polypeptide, alone or in combination with a soluble Ryk polypeptide, in culture, including ex vivo, or in vivo.

Accordingly, the present invention further relates to a method of ameliorating a neuronal disorder in a subject. In one embodiment, the method is performed by administering to a subject in need, a therapeutically effective amount of a Wnt polypeptide (e.g., Wnt3a, Wnt1, and/or Wnt4). The method can, but need not, further include administering a soluble Ryk polypeptide (e.g., extracelluiar domain of Ryk) to the subject. When administered, the soluble Ryk polypeptide can be administered at the same time as the Wnt polypeptide, in the same or a different formulation, or can be administered sequentially, e.g., by administering Wnt then Ryk, or Ryk then Wnt. In another embodiment, the method of ameliorating a neuronal disorder is performed by administering to the subject a therapeutically effective amount of a Wnt polypeptide, or a soluble Ryk polypeptide, or both a Wnt polypeptide and a Ryk polypeptide.

The subject to be treated according to the present methods can any subject having a neuronal disorder and containing neuronal or neuronal precursor cells in which neurite outgrowth can be induced by Wnt and/or soluble Ryk. Generally, the subject is a vertebrate subject such as a mammal (e.g., a domesticated animal, or a farm animal). In one aspect, the subject is a human subject having a neuronal disorder. Neuronal disorders amenable to amelioration using the present methods, include, for example, neurodegenerative diseases such as Parkinson's disease or Alzheimer's disease; congenital disorders; a nerve cell proliferative disorder (e.g., a neuroma, or neurofibromatosis); and traumatic nerve cell injuries such as spinal cord injuries.

The present invention relates to a method of inducing growth, proliferation, and/or differentiation of hematopoietic stem cells. Such a method can be performed, for example, by contacting at least one hematopoietic stem cell with a Wnt polypeptide and a soluble Ryk polypeptide. Wnt polypeptides and soluble Ryk polypeptides useful for the present methods can be those as disclosed herein, including, for example, a Wnt3a polypeptide and a peptide fragment of Ryk comprising the extracellular domain.

The present invention also relates to a composition that includes a soluble Ryk polypeptide. The soluble Ryk polypeptide can include, for example, an extracellular domain of a Ryk polypeptide that specifically binds Wnt and/or Frizzled. In one embodiment, an extracellular domain of Ryk polypeptide includes about amino acid residues 42-224 as set forth in SEQ ID NO: 14, or about amino acid residues 36-211 as set forth in SEQ ID NO: 16). A composition of the invention can further include a Wnt polypeptide, such as a Wnt 3a (e.g., SEQ ID NO:2, SEQ ID NO:4), Wnt1 (e.g., SEQ ID NO:6, SEQ ID NO:8), and Wnt4 (e.g., SEQ ID NO:10, SEQ ID NO:12) polypeptide, or a peptide functional fragment of a Wnt polypeptide that specifically binds Ryk and/or Frizzled. In one aspect, the composition is formulated such that it can be added to cells in culture without contaminating the culture. In another aspect, the composition is formulated for administration to a subject (e.g., human subject). Such a composition can be useful for practicing methods of the invention, including, for example, for stimulating growth, proliferation and/or differentiation of cells (e.g., neuronal cells, neuronal precursor cells, or hematopoietic cells), including for inducing neurite outgrowth. Further, the composition can be in a solid form (e.g., lyophilized), or can be is a solution, which can be an aqueous solution or a non-aqueous solution.

The present invention further relates to kits, which contain one or more compositions of the invention. For example, the kit can contain a soluble Ryk polypeptide and a Wnt polypeptide, which can be in one or separate compartments of the kit (e.g., separate tubes), and can be in a solid form or in solution. Where one or both of the soluble Ryk and Wnt component(s) is in a solid form, the kit can further contain one or more reagents for solubilizing the component(s). The soluble Ryk and/or a Wnt of the kit can further comprise a second component bound thereto, including, for example, a moiety such as a tag or detectable label, which can provide a means for detecting the presence of the Ryk and/or Wnt polypeptide, or the kit can contain one or a plurality of moieties that optionally can be linked to the Ryk and/or Wnt polypeptide.

The present invention further relates to a method of modulating an effect of Wnt on a cell. Such a method can be practiced, for example, by contacting the cell with a soluble Ryk polypeptide that selectively binds to Wnt and/or Frizzled, whereby selective binding of the soluble Ryk to Wnt and/or Frizzled alters Ryk mediated Wnt signal transduction in the cell. The soluble Ryk polypeptide (e.g., a Ryk extracellular domain) can affect Ryk mediated signal transduction, for example, by specifically interacting with Wnt and Frizzled, but not with Disheveled, thereby reducing or inhibiting Ryk mediated signal transduction via Disheveled in the cell. The cells in which an effect of Wnt can be modulated according to the present methods can be any cell in which Ryk is involved in the Wnt signal transduction pathway (e.g., neuronal and neuronal precursor, cancer cells, and hematopoietic cells).

The present invention also relates to screening assays useful for identifying a test agent that modulates Ryk activity. In one embodiment, the screening assay identifies an agent that modulates Ryk mediated signal transduction. Such a method can be practiced by contacting a sample containing Ryk and Frizzled with a test agent, under conditions suitable for binding of Wnt to Ryk and Frizzled, and detecting a change in Ryk mediated signal transduction due to selective binding of the agent to Ryk and Frizzled. In one aspect, the sample includes a cell that expresses an endogenous Ryk and Frizzled (e.g., a neuronal precursor cell), or that contains an exogenous Ryk and/or Frizzled, which can be expressed from a polynucleotide introduced into the cell (e.g., by transfection or transduction). In another aspect, the sample further includes a Wnt polypeptide.

In another embodiment, the screening assay identifies an agent that modulates a specific interaction of Ryk and Frizzled. Such a method can be performed by contacting a sample containing Ryk and Frizzled with a test agent, under conditions suitable for formation of a Ryk/Frizzled complex that mediates Ryk mediated signal transduction, and detecting a change in the complex in the presence of the test agent as compared to the absence of the test agent. In still another embodiment, the screening assay identifies an agent that modulates a specific interaction of Ryk and Disheveled. Such a method can be performed by contacting a sample having Ryk and Disheveled with a test agent, under conditions suitable for formation of a Ryk/Disheveled complex that mediates Ryk mediated signal transduction, and detecting a change in formation of the complex in the presence of the test agent as compared to complex formation in the absence of the test agent.

A change in Ryk mediated signal transduction can be detected, for example, by detecting a change in a downstream components of the Ryk mediated signal transduction pathway (e.g., by detecting a change in TCF activation in the presence of the test agent as compared to the absence of the test agent). A change in a Ryk/Dishevelled and/or Ryk/Frizzled complex can be detected using any method commonly used to examine complex formation, including complexes formed among proteins involved in a signal transduction pathway. For example, complex formation (and a change in complex formation) can be detected using a gel shift assay, a two hybrid assay, or a transcription based assay using a reporter construct such as a TCF gene regulatory element operatively linked to reporter gene (e.g., luciferase), expression of which is dependent on Ryk/Frizzled complex formation.

A test agent that can be examined according to the present methods can be any molecule that has or is suspected of having the ability to modulate Ryk activity, including agonist activity, antagonist activity, partial agonist activity, and the like. As such, the test agent can be any molecule of interest, including, for example, a peptide, polynucleotide, peptidomimetic, or small organic molecule. Further, the test agent can be one of a library of test agents, for example, a combinatorial library of test agents, which can be a random library, a biased library, or a variegated library, which can comprise test agents based on a general structure of a Ryk, Wnt, Frizzled, or Disheveled protein or on the structure of an agent identified as having Ryk modulating activity. The invention also provides an agent that modulates Ryk activity, wherein the agent is identified using a screening assay of the invention.

As disclosed herein, inhibition of Ryk activity can inhibit proliferation of cells that exhibit, or are predisposed to exhibiting, unregulated growth, including, for example, neoplastic cells (e.g., cancer cells). Accordingly, the present invention relates to a method of reducing or inhibiting proliferation of a cell that exhibits, or is predisposed to exhibiting, unregulated growth. Such a method can be performed, for example, by contacting the cell with an agent that selectively binds Ryk, whereby selective binding of the agent to Ryk reduces or inhibits Ryk mediated signal transduction in the cell, thereby inhibiting proliferation of the cell. In one embodiment, the agent reduces or inhibits Ryk mediated signal transduction by selectively binding the Ryk extracellular domain. Such an agent can act, for example, by altering the formation of Ryk complex involved in Ryk mediated signal transduction (e.g., the formation of a complex between Ryk and Wnt, and/or Ryk and Frizzled). In another embodiment, the agent reduces or inhibits Ryk mediated signal transduction by selectively binding the Ryk intracellular domain. Such an agent can act, for example, by altering the formation of a complex between Ryk and Disheveled. The agent can include any molecule capable of selectively binding Ryk, including, for example, a peptide, polynucleotide, peptidomimetic, or small organic molecule. In one embodiment, the agent is an agent identified using a screening assay of the invention. In another embodiment, the agent is an antibody that selectively binds Ryk.

The invention also relates to a method of inhibiting the proliferation of a cell that exhibits, or is predisposed to exhibiting, unregulated growth. Such a method can be practiced, for example, by contacting the cell with a soluble Ryk polypeptide, which selectively binds Wnt and/or Frizzled, whereby Ryk mediated signal transduction in the cell is reduced or inhibited, thereby inhibiting proliferation in the cell. The soluble Ryk polypeptide can be any soluble Ryk as disclosed herein (e.g., a polypeptide comprising about amino acid residues 42-224 as set forth in SEQ ID NO: 14, or about amino acid residues 36-211 as set forth in SEQ ID NO: 16).

The cell exhibiting, or predisposed to exhibiting, unregulated growth, generally is a vertebrate cell, including, for example, a mammalian cell (e.g., a human cell). Further, the cell can be a neoplastic cell such as a premalignant cell or a cancer cell (e.g., carcinoma cell or sarcoma cell). The cell exhibiting, or predisposed to exhibiting, unregulated growth can be contacted with the agent in culture (e.g., ex vivo) or in vivo in a subject. Where the agent is administered to a subject, it can be administered directly to the site of the target cells, or can be administered such that it diffuses or is transported (e.g., via the circulatory system) to the site of the cell (e.g., intrathecally, intraperitoneally, or intravenously). Accordingly, the present invention provides a method of ameliorating a cancer in a subject. In one embodiment, the method is performed by administering to the subject a therapeutically effective amount of an agent that selectively binds Ryk, whereby Ryk mediated signal transduction is reduced or inhibited. In another embodiment, the method is performed by administering to the subject a therapeutically effective amount of a soluble Ryk polypeptide, or an expressible polynucleotide encoding the soluble Ryk polypeptide.

### BRIEF DESCRIPTION OF THE DRAWINGS

FIG. 1 shows activation of a TCF-luciferase reporter by transfection with Ryk and treatment with Wnt3a conditioned medium.

FIG. 2 illustrates the schematic structure of lentiviral constructs expressing Ryk siRNA. The Ryk siRNA was expressed under the control of the human H1 promoter. GFP under the ubiquitin promoter was used as a control for infection.

FIG. 3 illustrates the endogenous Ryk mRNA levels in cells infected with lentivirus expressing Ryk siRNA.

FIG. 4 shows luciferase-reporter assay results indicating TCF activation for NFAT, NF-kappaB and TCF were cotransfected into 293T cells (dark bar) and Ryk siRNA (light bar) with dopamine receptor D2R, IKK β and Wnt-1, respectively. The results illustrates that Ryk is required for Wnt-1 induced TCF activation.

FIG. 5 illustrates that Ryk and Dishevelled synergistically activate the TCF luciferase reporter in 293T cells.

FIG. 6 shows TCF-luciferase reporter assay results for cells induced by Wnt3a and Ryk, cells co-transfected with Dishevelled-2 siRNA and Dishevelled-3, and dominant negative TCF-4. Co-transfection of Dishevelled-2 siRNA and Dishevelled-3 siRNAs blocks the activation of a TCF-luciferase reporter, as does dominant negative TCF-4. Dishevelled siRNAs and dominant negative TCF-4 were transfected with Ryk into 293T cells. The cells were treated with Wnt3a conditioned medium prior to luciferase reporter assay.

FIG. 7 illustrates Wnt induced synapse formation and neurite outgrowth in dorsal root ganglia (DRG) neurons. Results include quantification of neurite outgrowth in DRG explants treated with Wnt3a (Wnt3a) and those not treated (Control).

FIG. 8 illustrates Wnt induced synapse formation and neurite outgrowth in dorsal root ganglia (DRG) neurons. Results include quantification of neurite outgrowth in DRG explants treated with dilute conditioned Wnt3a media (Wnt3a) and those not treated (Control).

FIG. 9 show quantification of neurite number, indicating Wnt3a induced neurite outgrowth in DRG explants from wild type (light bars) and Ryk siRNA mice (dark bars). DRG explants were conditioned with Wnt3a media (Wnt3a) or not treated (Control).

FIG. 10 shows quantification of neurite length, indicating Wnt3a induced neurite outgrowth in DRG explants from wild type (WT) and Ryk siRNA mice (RykRNAi).

FIG. 11 illustrates quantification of neurite outgrowth in DRG explants from wild type (WT) and Ryk siRNA mice (Ryk RNAi) in response to nerve growth factor (NGF).

### DETAILED DESCRIPTION OF THE INVENTION

The present invention is based on the discovery that mammalian Ryk binds Wnt and functions in the Wnt signal transduction pathway. As disclosed herein, Ryk, which is a transmembrane protein, can function as a co-receptor with Frizzled to specifically bind a Wnt ligand, thereby mediating signal transduction due to Wnt (referred to herein as “Wnt mediate signal transduction” or “Ryk mediated signal transduction”). Further, Ryk can selectively bind the intracellular protein, Disheveled, which, in turn, activates downstream components of the canonical Wnt pathway, including TCF induced gene expression. As disclosed herein, Wnt can induce neurite outgrowth of neuronal and neuronal precursor cells, and Ryk is required for Wnt induced neurite outgrowth. As further disclosed herein, soluble forms of the Ryk polypeptide such as a polypeptide comprising the extracellular domain of Ryk can enhance the neurite outgrowth induced by Wnt. Remarkably, Ryk also can be targeted in cancer cells using, for example, small molecules, peptides (e.g., a soluble Ryk), or antibodies that reduce or inhibit the ability of Ryk to interact with Wnt, Frizzled, and/or Dishevelled, thereby reducing or inhibiting the growth and proliferation of the cancer cells.

The Wnt family of signaling molecules play an essential role in multiple, diverse developmental processes, including the regulation of cell proliferation, differentiation, and migration (Cadigan and Nusse, 1997; Moon et al., 2002; Peifer and Polakis, 2000; full citations follow Examples). The Wnt signaling pathway has an important role in the induction of interactions that regulate growth and differentiation, particularly during development and embryonic patterning. For example, gene targeting experiments suggest that the Wnt proteins are required for patterning of the central nervous system (Ikeya et al., 1997; McMahon and Bradley, 1990; McMahon et al., 1992; Thomas and Capecchi, 1990). Wnt is also involved in neural crest stem cell induction (Garcia-Castro et al., 2002; Lewis et al., 2004), neural precursor cell proliferation (Castelo-Branco et al., 2003; Chenn and Walsh, 2003; Ikeya et al., 1997), neurogenesis (Hari et al., 2002; Lee et al., 2004), axon guidance (Lyuksyutova et al., 2003; Yoshikawa et al., 2003), and synapse formation (Hall et al., 2000; Packard et al., 2002). Misregulation of the Wnt signaling pathway can cause developmental defects and is implicated in several diseases including neuronal diseases, as well as the growth and development of several human cancers (van Es et al., 2003).

The most well studied (canonical) Wnt signaling pathway is mediated by β-catenin (Willert and Nusse, 1998). In the absence of Wnt signaling, β-catenin is synthesized, but rapidly degraded due to phosphorylation by GSK30 (Aberle et al., 1997; Orford et al., 1997; Peifer et al., 1994; Salic et al., 2000; Yost et al., 1996). Wnt signaling inhibits the kinase activity of GSK30, allowing β-catenin to accumulate in the cytoplasm and to translocate to the nucleus. Nuclear β-catenin binds to members of the lymphoid enhancer binding factor/T cell-specific factor (LEF/TCF) family of transcription factors to activate the Wnt-target genes (Behrens et al., 1996; Huber et al., 1996; Molenaar et al., 1996). Wnt polypeptides are exemplified by human Wnt 3a (SEQ ID NO:2); mouse Wnt3a (SEQ ID NO:4); human Wnt1 (SEQ ID NO:6); mouse (SEQ ID NO:8); human Wnt4 (SEQ ID NO: 10); and mouse Wnt 4a (SEQ ID NO: 12). Nucleic acid molecules encoding these Wnt polynucleotides are disclosed herein as SEQ ID NOS: 1, 3, 5, 7, 9, and 11, respectively.

The canonical Wnt signal pathway requires both extracellular binding of Wnt and intracellular components for Wnt signal transduction. The extracellular Wnt polypeptides stimulate other cells through distinct receptors such as members of the Frizzled family and the co-receptor LDL receptor-related protein 5/6 (LRP5/6) (Bhanot et al., 1996; Tamai et al., 2000; Wehrli et al., 2000). LRP5/6 and Frizzled form a receptor-ligand complex with Wnt (Tamai et al., 2000). Additionally, the intracellular downstream adaptor protein, Dishevelled, also plays a key role in Wnt signaling.

Derailed in Drosophila is another receptor for Wnt (Yoshikawa et al., 2003). Derailed and its mammalian homolog, Ryk, are members of the atypical receptor tyrosine kinase family (Halford and Stacker, 2001). Ryk consists of an extracellular WIF domain, an intracellular atypical kinase domain, and a PDZ binding motif (Halford and Stacker, 2001). The kinase domain of Ryk is atypical in that it contains mutations in the evolutionarily conserved tyrosine kinase residues (Hovens et al., 1992; Yee et al., 1993) and lacks protein tyrosine kinase activity. The functions of Ryk have been studied in several model organisms including D. melanogaster, C. elegans, and M. musculus. The Drosophila Ryk homologue, Linotte or Derailed, was first identified as a gene involved in learning and memory (Dura et al., 1993; Dura et al., 1995). Furthermore, a mutation in the derailed gene causes defects in axon guidance (Bonkowsky et al., 1999; Callahan et al., 1995; Moreau-Fauvarque et al., 1998; Simon et al., 1998; Yoshikawa et al., 2003). Thus, the defects in learning and memory may be caused by the abnormal morphology of the central nervous system.

The C. elegans Ryk homologue, Lin-18, is required for establishing the polarity of the secondary vulval cell linage produced by a hypodermal blast cell. In vulva development, the Lin-18 mutant has a similar phenotype to the Lin-17 mutant (Sternberg and Horvitz, 1988), which the C. elegans homologue of Frizzled, suggesting a genetic interaction of Ryk and Frizzled. Ryk knockout mice die soon after birth and exhibit a complete cleft of the secondary palate plus a distinctive craniofacial appearance (Halford et al., 2000). This phenotype is also seen in EphB2/B3 knockout mice (Orioli et al., 1996), indicating that Ryk may be genetically linked to the Eph pathway, which is involved in development of the nervous system.

As disclosed herein, Ryk is an element of the Wnt-mediated signaling pathway that is involved in processes as diverse as neurite outgrowth, hematopoietic stem cell growth and differentiation, and cancer cell growth. More specifically, Ryk specifically binds Wnt polypeptides such as Wnt-1 and Wnt-3a through its extracellular WIF domain, and appears to form a co-receptor with Frizzled by binding the cysteine-rich domain of Frizzled. Ryk is shown to be required for Wnt-mediated TCF activation and Wnt-mediated neurite outgrowth, thus confirming that Ryk is a functional receptor for Wnt (Example 1). Further, Ryk is specifically binds Dishevelled, thereby providing a link between Wnt and the downstream scaffold protein Dishevelled (see Example 1). Transgenic mice expressing a Ryk siRNA exhibited defects in axon guidance, a phenotype that also is observed in Derailed mutant flies. Together, these results demonstrate that Ryk is a component of the Wnt signal transduction pathway.

Evidence suggests that the requirement of Ryk for Wnt signaling during development is context dependent. For example, while there are 19 Wnt genes in mammals, there is only one Ryk gene. Gene deletion experiments demonstrate that Wnt genes are required for a variety of developmental processes (Peifer and Polakis, 2000; Veeman et al., 2003; Wodarz and Nusse, 1998), whereas the Ryk gene is involved in only a few specific developmental procedures (Halford et al., 2000). This evidence implies that Ryk is involved in Wnt signaling in only a few specific cell types and, perhaps, in response to specific Wnt ligands. The expression pattern of Ryk and the different binding affinity of Ryk for various Wnt ligands can contribute to the specificity of Ryk function.

As disclosed herein, mammalian Ryk forms a complex with Wnt ligand and Frizzled (see Example 1). Ryk bound Frizzled in a ligand independent manner, indicating that Ryk functions as a co-receptor with Frizzled. While there is only one Ryk gene in mammals, there are three Ryk homologue genes in Drosophila—Derailed (Dr1), Doughnut (Dnt) and Derailed-2 (Drl-2). The molecular mechanism of each protein is different as Dnt can only partially rescue the muscle attachment defects in Drl mutant (Oates et al., 1998). Although Derailed functions independently of Frizzled for commissural axon guidance (Lyuksyutova et al., 2003), the present results suggest that Dnt or Drl-2 also can interact with Frizzled.

As further disclosed herein, Ryk associated with Dishevelled, and this association required the PDZ binding motif of Ryk. It was suggested previously that the PDZ domain of Dishevelled binds to a sequence of Frizzled at the C-terminus (Wong et al., 2003); however, this interaction is relatively weak. The disclosed interaction of Ryk with Wnt extracellularly and with Dishevelled intracellularly provides a previously undescribed link between Wnt and Dishevelled, and can explain the weak binding previously observed between Dishevelled and Frizzled.

As shown in Example 1, a functional Ryk polypeptide is required for TCF activation. An RNAi directed at the Ryk gene in 293T cells inhibited the TCF activation induced by Wnt-1, indicating that Ryk is required for the TCF pathway in this situation. While anti-Ryk siRNA blocked the activation of a TCF-luciferase reporter, overexpression of Ryk only modestly activated it, suggesting that endogenous Ryk levels are near saturating level for activation of the TCF pathway. There are two pathways that regulate the TCF-driven target gene expression. One pathway acts through the accumulation and nuclear translocation of β-catenin, which then binds to TCF and changes TCF from a repressor to an activator (Behrens et al., 1996; Huber et al., 1996; Molenaar et al., 1996). The second pathway is less well characterized but is mediated by a nemo-like kinase (NLK) that inhibits the TCF transactivation (Ishitani et al., 2003; Ishitani et al., 1999; Smit et al., 2004). The fact that Dishevelled is required in TCF activation induced by Ryk and Wnt3a indicates that Ryk is involved in the canonical Wnt pathway leading to TCF activation.

Wnt and Ryk function in inducing neurite outgrowth and axon guidance. Ryk siRNA mice had defects in axon guidance of craniofacial motor nerves, ophthalmic nerves, and other nerves, indicating an essential role of Ryk in axon guidance. Although there is no obvious deficiency in DRG neurite outgrowth in Ryk siRNA transgenic mice, DRG explants isolated from Ryk siRNA mice exhibit defects in neurite outgrowth in response to Wnt3a stimulation. As mentioned, the lack of deficiency in DRG neurite outgrowth in Ryk siRNA mice was probably because NGF and other growth factors are also involved in inducing neurite outgrowth in vivo. The fact that the Wnt-3a-induced neurite outgrowth of DRG explants is inhibited in Ryk siRNA mice provides strong evidence that there is a functional interaction between Wnt and Ryk in neurite outgrowth. Accordingly, the present invention provides a method of inducing neurite outgrowth by contacting a neuronal cell or a neuronal precursor cell with a Wnt polypeptide.

The term “neurite outgrowth” refers to the growing out and formation of elongated, membrane-enclosed protrusions of neuron cytoplasm, which form axons and dendrites that connect with other neurons. Neurites are critical for intercellular communication and the process of neurite outgrowth is essential in neural development and regeneration. Neurite outgrowth is important during development, and is very slow or non-existent in adults. As a result, nerve injuries can be slow to heal and, in many cases, are never repaired. The methods of the invention provide a means to facilitate nerve injuries by inducing neurite outgrowth using, for example, a Wnt polypeptide and/or a soluble Ryk polypeptide, or a peptide function fragment of these polypeptides.

Wnt polypeptides useful for inducing neurite outgrowth include polypeptides and peptide functional fragments that selectively bind Ryk and Frizzled. Such Wnt polypeptides are exemplified by human and mouse Wnt 3a (e.g., SEQ ID NO:2, SEQ ID NO:4), Wnt1 (e.g., SEQ ID NO:6, SEQ ID NO:8), and Wnt4 (e.g., SEQ ID NO:10, SEQ ID NO:12). As used herein, the term “selectively binds” or “specifically binds” or the like refers to two or more molecules that form a complex that is relatively stable under physiologic conditions or conditions suitable for binding. The term is used herein in reference to various interactions, including, for example, the association of an agent or ligand and one or more polypeptides involved in Ryk mediated signal transduction, or the association between two polypeptides involved in Ryk mediated signal transduction(e.g., Wnt and Ryk; Ryk and Disheveled; Ryk and Frizzled; and Ryk, Wnt, and Frizzled). Two molecules that specifically associate can be characterized by a dissociation constant of at least about 1×10⁻⁶ M, generally at least about 1×10⁻⁷ M, usually at least about 1×10⁻⁸ M, and particularly at least about 1×10⁻⁹ M or 1×10⁻¹⁰ M or greater.

For purposes of the present invention, selective binding can occur, and is stable, under physiological conditions and conditions that mimic physiological conditions, including, for example, conditions that occur in a living individual such as a human or other vertebrate or invertebrate, and conditions that occur in a cell culture such as used for maintaining mammalian cells or cells from another vertebrate organism or an invertebrate organism. Various examples of conditions suitable for selective binding of components of the Ryk signal transduction pathway, as well as methods of determining such conditions, are disclosed herein (see Examples 1 and 2), or otherwise known in the art.

As used herein, the term “functional fragment” or “peptide functional fragment”, when used in reference to a Wnt polypeptide, means a peptide portion of a Wnt polypeptide that can selectively bind Ryk and/or Frizzled and induce Ryk mediated signal transduction. Methods for identifying a peptide functional fragment of Wnt are disclosed herein, or otherwise known in the art. For example, a functional fragment of Wnt that selectively binds Ryk and/or Frizzled can be identified using any of various assays known to be useful for identifying specific protein-protein interactions. Such assays include, for example, methods of gel electrophoresis (e.g., gel mobility shift assays), affinity chromatography, the two hybrid system of Fields and Song (Nature 340:245-246, 1989; see, also, U.S. Pat. No.5,283,173; Fearon et al., Proc. Natl. Acad. Sci., USA 89:7958-7962, 1992; Chien et al., Proc. Natl. Acad. Sci. USA 88:9578-9582, 1991; Young, Biol. Reprod. 58:302-311(1998), each of which is incorporated herein by reference), the reverse two hybrid assay (Leanna and Hannink, Nucl. Acids Res. 24:3341-3347, 1996, which is incorporated herein by reference), the repressed transactivator system (U.S. Pat. No.5,885,779, which is incorporated herein by reference), and the like (see, for example, Mathis, Clin. Chem. 41:139-147, 1995 Lam, Anticancer Drug Res. 12:145-167, 1997; Phizicky et al., Microbiol. Rev. 59:94-123, 1995; each of which is incorporated herein by reference). A functional fragment of a Wnt polypeptide also can be identified using methods of molecular modeling.

It should be recognized that such methods, including two hybrid assays and molecular modeling methods, also can be used to identify other selectively binding molecules encompassed within the present invention. For example, a method such as the two hybrid assay can be used to identify a peptide functional fragment of a Ryk polypeptide that selectively binds Wnt and/or Frizzled, including, for example, a soluble Ryk polypeptide (e.g., a peptide comprising the Ryk extracellular domain), as well as a peptide functional fragment of a Ryk polypeptide that selectively binds Dishevelled (e.g., peptide comprising the Ryk intracellular domain). As disclosed herein, such assays also can be used to detect changes in a complex formation (e.g., a Ryk/Frizzled complex) and, therefore, can be useful in the screening assays of the invention to identify agents that modulate a specific interaction of Ryk and Frizzled.

In addition to using a Wnt polypeptide to induce neurite outgrowth, the invention provides a method of inducing neurite outgrowth by further contacting a cell with a soluble Ryk polypeptide. As used herein, the term “soluble Ryk” refers to a Ryk polypeptide that lacks all or a sufficient portion of the Ryk transmembrane domain such that, when it is expressed in a cell, it does not become membrane bound. A soluble Ryk polypeptide useful in the present invention is characterized, in part, in that it assumes an appropriate conformation under aqueous conditions such that is can specifically bind a polypeptide that wild type Ryk can specifically bind. As such, a soluble Ryk polypeptide comprising the Ryk extracellular domain can retain the ability to selectively bind and form a complex with a membrane bound Frizzled polypeptide; and/or with a Wnt ligand. Such a soluble Ryk polypeptide is exemplified by about amino acid residues 42-224 as set forth in SEQ ID NO:14, or about amino acid residues 36-211 as set forth in SEQ ID NO:16. Similarly, a soluble Ryk polypeptide comprising the Ryk intracellular domain can retain the ability to selectively bind and form a complex with a Dishevelled polypeptide. An intracellular domain of a Ryk polypeptide is exemplified by about amino acid residues 248-605 as set forth in SEQ ID NO:14, or about amino acid residues 235-595 as set forth in SEQ ID NO:16.

As used herein, the term “about”, when used in reference to the amino acid residues of polypeptide, can lack or contain one or a few ( 2, 3, 4, 5, etc.) amino acid residues from the N-terminus and/or C-terminus, provided the polypeptide lacking or containing the one or few additional amino acid residues maintains the function of the specified polypeptide. As such, it should be recognized that the term “about” is used in this context because the loss or addition of a few amino acid residues at a terminus of a polypeptide generally does not substantially affect the function of the polypeptide. By way of example, reference to a soluble Ryk having a sequence of about amino acid residues 1 to 200 of SEQ ID NO:16 includes a Ryk polypeptide having a sequence of amino acid residues 2 to 194, or amino acid residues 5 to 205. Preferably, the N-terminus begins one of a amino acid residues 1 to 6 and the C-terminus ends at one of amino acid residue 197 to 203. It should further be recognized that a polypeptide useful in the compositions and/or methods of the invention can be a fusion protein (e.g., a tagged polypeptide, or a chimeric polypeptide comprising a first and second (or more) polypeptide(s)). Such fusion proteins are not encompassed within the meaning of the term “about” as defined herein.

The methods of the invention provide a means to modulate the growth, proliferation, and/or differentiation of cells, including, for example, to induce neurite outgrowth of neuronal and neuronal precursor cells and to reduce or inhibit the proliferation of cancer cells. As used herein, the term “modulate” means “increased” or “reduced or inhibited”. The terms “increase” and “reduce or inhibit” are used in reference to a baseline level of the specified activity (e.g., cell growth, Ryk activity, and Wnt mediated signal transduction”), which can be the level of the specified activity in the absence of an agent that has the modulating activity, or the level of the specified activity with respect to a corresponding normal cell. For example, the Ryk mediated signal transduction pathway exhibits a particular activity in a neuronal cell, and, upon further contacting the neuronal cell with a soluble Ryk polypeptide comprising the extracellular domain, Ryk mediated signal transduction activity is increased, resulting in neurite outgrowth. As such, a soluble Ryk polypeptide is an agent useful for increasing neurite outgrowth.

In another example, specified cancer cells exhibit a certain (baseline) level of cell proliferation, wherein, upon contact with a soluble Ryk polypeptide comprising the Ryk extracellular domain, proliferation of the cancer cells is reduced or inhibited with respect the level of proliferation in the absence of the soluble Ryk. It should be recognized that the terms “reduce or inhibit” are used together herein because, in some cases, the level of Wnt mediated signal transduction, for example, can be reduced below a level that can be detected by a particular assay. As such, it may not be determinable using such an assay as to whether a low level of Wnt mediated signal transduction remains, or whether the signal transduction is completely inhibited. Nevertheless, it will be clearly determinable that at least a decrease in the level of signal transduction occurs.

Cells amenable to manipulation according to the present methods include any cells in which Ryk constitutes a required component of Wnt mediated signal transduction (i.e., Ryk mediated signal transduction), particularly vertebrate cells, including mammalian cells (e.g., human cells). Further, the cells can be normal cells or cells that are diseased, damaged, or exhibit or predisposed to exhibiting unregulated growth. As such, the cells can be cells obtained from a normal, healthy individual, cells from a subject having a neuronal disorder, which can be a disorder associated with traumatic nerve injury or a neurodegenerative disease, or can be cells from a subject having a cancer, including normal cells and/or cancer cells from such a subject. As disclosed herein, such cells can be contacted ex vivo and/or in vivo with a composition to achieve a desired effect (e.g., a Wnt polypeptide, a soluble Ryk polypeptide, an anti-Ryk antibody, or an agent identified according to a screening assay of the invention).

Accordingly, in one embodiment, the invention provides a method of ameliorating a neuronal disorder in a subject. As used herein, the term “ameliorate” means that signs or symptoms associated with the condition are lessened. Examples of neuronal disorders amenable to treatment according to the methods of the invention include, but are not limited to, neurodegenerative disorders such as Alzheimer's disease, Parkinson's disease, and Huntington's disease, and neuronal disorders associated with stroke or an ischemic event, epilepsy, and traumatic nerve injuries such as brain and spinal cord injuries. As such, the signs or symptoms to be monitored will be characteristic of the particular neuronal disorder being treated, and will be well known to the skilled clinician, as will the methods for monitoring the signs and conditions. For example, where the neuronal disorder is Alzheimer's disease, the skilled clinician can monitor indicia of cognitive function including, for example, memory recall, counting, language skills, and the like in the subject. Where the neuronal disorder is traumatic nerve injury, the clinician can monitor the subject's motor function, including, for example, strength and dexterity.

A method of ameliorating a neuronal disorder in a subject can be practiced by administering to the subject a therapeutically effective amount of a Wnt polypeptide or functional fragment thereof, a soluble Ryk polypeptide, or a combination thereof. As used herein, the term “therapeutically effective amount” means an amount of the therapeutic agent being administered that elicits the biological or medical response of a cell, tissue, system, or subject that is being sought by the researcher, veterinarian, medical doctor or other clinician. The biological and/or medical response can include, for example, modulating Ryk mediated signal transduction, induction of neurite outgrowth, restoration or maintenance of neurodegeneration, prevention of neurodegeneration, improvement or maintenance of cognitive function or motor function, and, in aspects of the invention, reduction of tumor burden and/or rate of tumor cell growth, and reduction of morbidity and/or mortality.

In another embodiment, the invention provides a method inhibiting the proliferation of cells that exhibit, or are predisposed to exhibiting, unregulated growth by reducing or inhibiting Ryk activity in the cells. As used herein, the term “Ryk activity” means the ability of Ryk to specifically bind Wnt, Frizzled, and/or Dishevelled. Generally, an agent that modulates Ryk activity will correspondingly modulate Ryk mediated signal transduction activity (e.g., reduce or inhibit). As used herein, the term “Ryk mediated signal transduction activity” refers to the signaling pathway that is initiated by the binding Wnt (or a Wnt mimic—e.g., an agonist or antagonist) to Ryk or to Ryk and Frizzled, is transmitted via Disheveled, and ends in expression of one or more genes (e.g., TCF). The term “Wnt mediated signal transduction activity” also is used herein to refer to pathways that are initiated by Wnt binding to Frizzled, but not necessarily Ryk. As such, the Ryk mediated signal transduction can be considered a subset of the Wnt mediated signal transduction pathways.

A cell that exhibits, or is predisposed to exhibiting, unregulated growth and, therefore, amenable to treatment according to the present methods can be a neoplastic cell, which can be, for example, a premalignant cell, or can be a cancer cell, for example, a carcinoma cell or a sarcoma cell. In one embodiment, the method is performed by contacting the cell with an agent that selectively binds Ryk, whereby selective binding of the agent to Ryk inhibits Ryk mediated signal transduction in the cell, thereby inhibiting proliferation of the cell. The agent can be any agent that selectively binds Ryk and inhibits Ryk mediated signal transduction, including, for example, a peptide, polynucleotide, peptidomimetic, or small organic molecule that interferes with the formation of Ryk complex (e.g., Ryk/Frizzled; Wnt/Ryk; Wnt/Ryk/Frizzled; Ryk/Dishevelled). As such, the agent can act, for example, by binding the Ryk extracellular domain (e.g., an anti-Ryk antibody) or the Ryk intracellular domain (e.g., a soluble Ryk peptide comprising the Ryk intracellular domain). In another embodiment, the agent comprises a soluble Ryk extracellular domain, and lacks at least the peptide portion of the Ryk intracellular domain that allows selective binding of Ryk to Dishevelled, wherein the Ryk extracellular domain can selectively bind Wnt and/or Frizzled, but is not capable of transmitting a signal to Dishevelled.

In one aspect, the method of reducing or inhibiting unregulated growth of a cell, or preventing unregulated growth of a cell predisposed to exhibiting unregulated growth can be performed by contacting the cell with an antibody that selectively binds Ryk. As used herein, the term “antibody” is used in its broadest sense to include polyclonal and monoclonal antibodies, as well as antigen binding fragments of such antibodies. Similarly to as discussed above, the term “binds specifically” or “specific binding activity,” when used in reference to an antibody, means that the interaction of the antibody and a particular epitope has a dissociation constant of at least about 1×10⁻⁶, generally at least about 1×10⁻⁷, usually at least about 1×10⁻⁸, and particularly at least about 1×10⁻⁹ or less. Antibody fragments such as Fab, F(ab′)₂, Fd and Fv fragments of an antibody that retain specific binding activity for an epitope of a polypeptide, are included within the definition of an antibody.

An antibody useful in the present compositions and passive immunization methods includes naturally occurring antibodies as well as non-naturally occurring antibodies, including, for example, single chain antibodies, chimeric, bifunctional and humanized antibodies, as well as antigen-binding fragments thereof. Such non-naturally occurring antibodies can be constructed using solid phase peptide synthesis, can be produced recombinantly or can be obtained, for example, by screening combinatorial libraries consisting of variable heavy chains and variable light chains (see Huse et al., Science 246:1275-1281 (1989), which is incorporated herein by reference). These and other methods of making, for example, chimeric, humanized, CDR-grafted, single chain, and bifunctional antibodies are well known to those skilled in the art (Winter and Harris, Immunol. Today 14:243-246, 1993; Ward et al., Nature 341:544-546, 1989; Harlow and Lane, Antibodies: A laboratory manual (Cold Spring Harbor Laboratory Press, 1988); Hilyard et al., Protein Engineering: A practical approach (IRL Press 1992); Borrabeck, Antibody Engineering, 2d ed. (Oxford University Press 1995); each of which is incorporated herein by reference).

Methods for raising polyclonal antibodies, for example, in a rabbit, goat, mouse or other mammal, are well known in the art (see, for example, Green et al., “Production of Polyclonal Antisera,” in Immunochemical Protocols (Manson, ed., Humana Press 1992), pages 1-5; Coligan et al., “Production of Polyclonal Antisera in Rabbits, Rats, Mice and Hamsters,” in Curr. Protocols Immunol. (1992), section 2.4.1; each or which is incorporated herein by reference). In addition, monoclonal antibodies can be obtained using methods that are well known and routine in the art (Harlow and Lane, supra, 1988). Methods of preparing monoclonal antibodies well known (see, for example, Kohler and Milstein, Nature 256:495, 1975, which is incorporated herein by reference; see, also, Coligan et al., supra, 1992, see sections 2.5.1-2.6.7; Harlow and Lane, supra, 1988). Monoclonal antibodies can be isolated and purified from hybridoma cultures by a variety of well established techniques, including, for example, affinity chromatography with Protein-A SEPHAROSE gel, size exclusion chromatography, and ion exchange chromatography (Coligan et al., supra, 1992, see sections 2.7.1-2.7.12 and sections 2.9.1-2.9.3; see, also, Barnes et al., “Purification of Inuunoglobulin G (IgG),” in Meth. Molec. Biol. 10:79-104 (Humana Press 1992), which is incorporated herein by reference).

The antibodies also can be derived from human antibody fragments isolated from a combinatorial immunoglobulin library (see, for example, Barbas et al., METHODS: A Companion to Methods in Immunology 2:119, 1991; Winter et al., Ann. Rev. Immunol. 12:433, 1994; each of which is incorporated herein by reference). Cloning and expression vectors that are useful for producing a human immunoglobulin phage library can be obtained, for example, from STRATAGENE Cloning Systems (La Jolla, Calif.). An antibody also can be derived from a human monoclonal antibody. Such antibodies are obtained from transgenic mice that have been “engineered” to produce specific human antibodies in response to antigenic challenge. Methods for obtaining human antibodies from transgenic mice are described, for example, by Green et al., Nature Genet. 7:13, 1994; Lonberg et al., Nature 368:856, 1994; and Taylor et al., Int. Immunol. 6:579, 1994; each of which is incorporated herein by reference.

In another aspect, the method of reducing or inhibiting unregulated growth of a cell, or preventing unregulated growth of a cell predisposed to exhibiting unregulated growth utilizes active immunization. Such a method can be performed by contacting the cell with peptide comprising an epitope of a Ryk polypeptide, wherein an immune response stimulated against the peptide epitope is crossreactive with Ryk expressed by the target cells. Where such a peptide is non-immunogenic, it can be made immunogenic by coupling the hapten to a carrier molecule such as bovine serum albumin (BSA) or keyhole limpet hemocyanin (KLH), or by expressing the peptide epitope as a fusion protein. Various other carrier molecules and methods for coupling a hapten to a carrier molecule are well known in the art (see, for example, by Harlow and Lane, supra, 1988), as are adjuvants that can be useful for enhancing the immune response against the target Ryk polypeptide. Accordingly, the invention provides a cancer vaccine comprising a Ryk epitope comprising a peptide portion of a Ryk extracellular domain.

In still another aspect, the method of reducing or inhibiting unregulated growth of a cell, or preventing unregulated growth of a cell predisposed to exhibiting unregulated growth can be performed by contacting the cell with a soluble Ryk polypeptide, which selectively binds Wnt and/or Frizzled and alters Ryk mediated signal transduction, thereby reducing or inhibiting proliferation in the cell. In one aspect, the soluble Ryk polypeptide generally includes at least a Wnt and/or Frizzled binding portion of the Ryk extracellular domain of a Ryk polypeptide, which comprises about amino acid residues 42-224 of human Ryk (SEQ ID NO:14) or about amino acid residues 36-211 of murine Ryk (SEQ ID NO:16). In another aspect, the soluble Ryk polypeptide is encoded by an expressible polynucleotide, wherein the polynucleotide is contacted with a cell under conditions suitable for introduction of the polynucleotide into the cell and expression of the encoded soluble Ryk. The cell into which the expressible polynucleotide is introduced can, but need not be, the target cell (i.e., the cell exhibiting or predisposed to exhibiting unregulated growth), provided that when the cell is not the target cell, the expressed soluble Ryk is secreted, actively or passively, from the cell such that it can contact the target cell and effect its action. The present invention also provides a method of ameliorating a cancer in a subject using a method as disclosed above for reducing or inhibiting unregulated growth of a cell exhibiting or predisposed to exhibiting the unregulated growth. As such, the method can be performed using active and/or passive immunization such that anti-Ryk antibodies can reduce or inhibit Ryk activity in the target cell, and/or using a soluble Ryk polypeptide and/or expressible polynucleotide encoding the soluble Ryk.

The term “polynucleotide” is used broadly herein to mean a sequence of two or more deoxyribonucleotides or ribonucleotides that are linked together by a phosphodiester bond. As such, the term “polynucleotide” includes RNA and DNA, which can be a gene or a portion thereof, a cDNA, a synthetic polydeoxyribonucleic acid sequence, or the like, and can be single stranded or double stranded, as well as a DNA/RNA hybrid. Furthermore, the term “polynucleotide” as used herein includes naturally occurring nucleic acid molecules, which can be isolated from a cell, as well as synthetic molecules, which can be prepared, for example, by methods of chemical synthesis or by enzymatic methods such as by the polymerase chain reaction (PCR). In various embodiments, the polynucleotide can contain nucleoside or nucleotide analogs, or a backbone bond other than a phosphodiester bond (see above).

In general, the nucleotides comprising a polynucleotide are naturally occurring deoxyribonucleotides, such as adenine, cytosine, guanine or thymine linked to 2′-deoxyribose, or ribonucleotides such as adenine, cytosine, guanine or uracil linked to ribose. However, a polynucleotide also can contain nucleotide analogs, including non-naturally occurring synthetic nucleotides or modified naturally occurring nucleotides. Such nucleotide analogs are well known in the art and commercially available, as are polynucleotides containing such nucleotide analogs (Lin et al., Nucl. Acids Res. 22:5220-5234 (1994); Jellinek et al., Biochemistry 34:11363-11372 (1995); Pagratis et al., Nature Biotechnol. 15:68-73 (1997), each of which is incorporated herein by reference).

The covalent bond linking the nucleotides of a polynucleotide generally is a phosphodiester bond. However, the covalent bond also can be any of numerous other bonds, including a thiodiester bond, a phosphorothioate bond, a peptide-like bond or any other bond known to those in the art as useful for linking nucleotides to produce synthetic polynucleotides (see, for example, Tam et al., Nucl. Acids Res. 22:977-986 (1994); Ecker and Crooke, BioTechnology 13:351360 (1995), each of which is incorporated herein by reference). The incorporation of non-naturally occurring nucleotide analogs or bonds linking the nucleotides or analogs can be particularly useful where the polynucleotide is to be exposed to an environment that can contain a nucleolytic activity, including, for example, a tissue culture medium or upon administration to a living subject, since the modified polynucleotides can be less susceptible to degradation.

A polynucleotide comprising naturally occurring nucleotides and phosphodiester bonds can be chemically synthesized or can be produced using recombinant DNA methods, using an appropriate polynucleotide as a template. In comparison, a polynucleotide comprising nucleotide analogs or covalent bonds other than phosphodiester bonds generally will be chemically synthesized, although an enzyme such as T7 polymerase can incorporate certain types of nucleotide analogs into a polynucleotide and, therefore, can be used to produce such a polynucleotide recombinantly from an appropriate template (Jellinek et al., supra, 1995).

Where a polynucleotide encodes a peptide, for example, a soluble Ryk polypeptide, the coding sequence can be contained in a vector and is operatively linked to appropriate regulatory elements, including, if desired, a tissue specific promoter or enhancer. The encoded polypeptide can be further operatively linked, for example, to a peptide tag such as a His-6 tag or the like, which can facilitate identification of expression of the polypeptide in the target cell. A polyhistidine tag peptide such as His-6 can be detected using a divalent cation such as nickel ion, cobalt ion, or the like. Additional peptide tags include, for example, a FLAG epitope, which can be detected using an anti-FLAG antibody (see, for example, Hopp et al., BioTechnology 6:1204 (1988); U.S. Pat. No. 5,011,912, each of which is incorporated herein by reference); a c-myc epitope, which can be detected using an antibody specific for the epitope; biotin, which can be detected using streptavidin or avidin; and glutathione S-transferase, which can be detected using glutathione. Such tags can provide the additional advantage that they can facilitate isolation of the operatively linked polypeptide or peptide agent, for example, where it is desired to obtain, for example, a substantially purified soluble Ryk polypeptide.

As used herein, the term “operatively linked” or “operatively associated” means that two or more molecules are positioned with respect to each other such that they act as a single unit and effect a function attributable to one or both molecules or a combination thereof. For example, a polynucleotide sequence encoding a soluble Ryk polypeptide can be operatively linked to a regulatory element, in which case the regulatory element confers its regulatory effect on the polynucleotide similarly to the way in which the regulatory element would effect a polynucleotide sequence with which it normally is associated with in a cell. A first polynucleotide coding sequence also can be operatively linked to a second (or more) coding sequence such that a chimeric polypeptide can be expressed from the operatively linked coding sequences. The chimeric polypeptide can be a fusion polypeptide, in which the two (or more) encoded peptides are translated into a single polypeptide, i.e., are covalently bound through a peptide bond; or can be translated as two discrete peptides that, upon translation, can operatively associate with each other to form a stable complex.

A chimeric polypeptide generally demonstrates some or all of the characteristics of each of its peptide components. As such, a chimeric polypeptide can be particularly useful in performing methods of the invention, as disclosed herein. For example, a method of the invention can be practiced by introducing ex vivo into cells of a subject to be treated, or into cells that are haplotype matched to the subject, a polynucleotide encoding soluble Ryk operatively linked to a signal peptide that directs secretion of the chimeric polypeptide from the cell. The cell then can be administered to the subject, wherein, upon expression of the chimeric polypeptide, the signal peptide directs secretion of the polypeptide from the cell and the soluble Ryk component of the chimeric polypeptide can effect its Ryk inhibitory action upon contact with a target cell.

A chimeric polypeptide also can include a cell compartmentalization domain. Cell compartmentalization domains are well known and include, for example, a plasma membrane localization domain, a nuclear localization signal, a mitochondrial membrane localization signal, an endoplasmic reticulum localization signal, or the like (see, for example, Hancock et al., EMBO J. 10:4033-4039, 1991; Buss et al., Mol. Cell. Biol. 8:3960-3963, 1988; U.S. Pat. No. 5,776,689 each of which is incorporated herein by reference). Such a domain can be useful to target a polypeptide agent to a particular compartment in the cell, or, as discussed above, to target the polypeptide for secretion from a cell.

A polynucleotide useful for performing a method of the invention also can act directly with a Ryk polypeptide expressed on a target cell to reduce or inhibit the Ryk activity. Such polynucleotide agents, which can interact specifically with a target Ryk polypeptide, can be made and identified using methods well known in the art (see, for example, O'Connell et al., Proc. Natl. Acad. Sci., USA 93:5883-5887, 1996; Tuerk and Gold, Science 249:505-510, 1990; Gold et al., Ann. Rev. Biochem. 64:763-797, 1995; each of which is incorporated herein by reference).

A polynucleotide useful in performing a method of the invention, can be contained in a vector, which can facilitate manipulation of the polynucleotide, including introduction of the polynucleotide into a target cell. The vector can be a cloning vector, which is useful for maintaining the polynucleotide, or can be an expression vector, which contains, in addition to the polynucleotide, regulatory elements useful for expressing the polynucleotide and, where the polynucleotide encodes a polypeptide, for expressing the encoded peptide in a particular cell. An expression vector can contain the expression elements necessary to achieve, for example, sustained transcription of the encoding polynucleotide, or the regulatory elements can be operatively linked to the polynucleotide prior to its being cloned into the vector.

An expression vector (or the polynucleotide) generally contains or encodes a promoter sequence, which can provide constitutive or, if desired, inducible or tissue specific or developmental stage specific expression of the encoding polynucleotide, a poly-A recognition sequence, and a ribosome recognition site or internal ribosome entry site, or other regulatory elements such as an enhancer, which can be tissue specific. The vector also can contain elements required for replication in a prokaryotic or eukaryotic host system or both, as desired. Such vectors, which include plasmid vectors and viral vectors such as bacteriophage, baculovirus, retrovirus, lentivirus, adenovirus, vaccinia virus, semliki forest virus and adeno-associated virus vectors, are well known and can be purchased from a commercial source (Promega, Madison Wis.; Stratagene, La Jolla Calif.; GIBCO/BRL, Gaithersburg Md.) or can be constructed by one skilled in the art (see, for example, Meth. Enzymol., Vol. 185, Goeddel, ed. (Academic Press, Inc., 1990); Jolly, Canc. Gene Ther. 1:51-64, 1994; Flotte, J. Bioenerg. Biomemb. 25:37-42, 1993; Kirshenbaum et al., J. Clin. Invest. 92:381-387, 1993; each of which is incorporated herein by reference). A tetracycline (tet) inducible promoter is an example of a promoter that can be useful for driving expression of a polynucleotide, wherein, upon administration of tetracycline, or a tetracycline analog, to a subject containing a polynucleotide operatively linked to a tet inducible promoter, expression of the encoded polypeptide is induced.

The polynucleotide also can be operatively linked to tissue specific regulatory element, for example, a neuronal cell specific regulatory element, such that expression of an encoded peptide is restricted to neuronal cells in an individual, or to neuronal cells in a mixed population of cells in culture, for example, an organ culture.

Viral expression vectors can be particularly useful for introducing a polynucleotide into a cell, particularly a cell in a subject. Viral vectors provide the advantage that they can infect host cells with relatively high efficiency and can infect specific cell types. For example, a polynucleotide encoding a soluble Ryk polypeptide can be cloned into a baculovirus vector, which then can be used to infect an insect host cell, thereby providing a means to produce large amounts of the soluble Ryk. The viral vector also can be derived from a virus that infects cells of an organism of interest, for example, vertebrate host cells such as mammalian, avian or piscine host cells. Viral vectors can be particularly useful for introducing a polynucleotide useful in performing a method of the invention into a target cell. Viral vectors have been developed for use in particular host systems, particularly mammalian systems and include, for example, retroviral vectors, other lentivirus vectors such as those based on the human immunodeficiency virus (HIV), adenovirus vectors, adeno-associated virus vectors, herpesvirus vectors, vaccinia virus vectors, and the like (see Miller and Rosman, BioTechniques 7:980-990, 1992; Anderson et al., Nature 392:25-30 Suppl., 1998; Verma and Somia, Nature 389:239-242, 1997; Wilson, New Engl. J. Med. 334:1185-1187 (1996), each ofwhich is incorporated herein by reference).

A polynucleotide, which can be contained in a vector, can be introduced into a cell by any of a variety of methods known in the art (Sambrook et al., Molecular Cloning: A laboratory manual (Cold Spring Harbor Laboratory Press 1989); Ausubel et al., Current Protocols in Molecular Biology, John Wiley and Sons, Baltimore, Md. (1987, and supplements through 1995), each of which is incorporated herein by reference). Such methods include, for example, transfection, lipofection, microinjection, electroporation and, with viral vectors, infection/transduction; and can include the use of liposomes, microemulsions or the like, which can facilitate introduction of the polynucleotide into the cell and can protect the polynucleotide from degradation prior to its introduction into the cell. The selection of a particular method will depend, for example, on the cell into which the polynucleotide is to be introduced, as well as whether the cell is isolated in culture, or is in a tissue or organ in culture or in situ.

Introduction of a polynucleotide into a cell by infection with a viral vector is particularly advantageous in that it can efficiently introduce the nucleic acid molecule into a cell ex vivo or in vivo (see, for example, U.S. Pat. No. 5,399,346, which is incorporated herein by reference). Moreover, viruses are very specialized and can be selected as vectors based on an ability to infect and propagate in one or a few specific cell types. Thus, their natural specificity can be used to target the nucleic acid molecule contained in the vector to specific cell types. As such, a vector based on a herpesvirus can be used to infect neuronal cells, a vector based on an HIV can be used to infect T cells, a vector based on an adenovirus can be used, for example, to infect respiratory epithelial cells, and the like. Other vectors, such as adeno-associated viruses can have greater host cell range and, therefore, can be used to infect various cell types, although viral or non-viral vectors also can be modified with specific receptors or ligands to alter target specificity through receptor mediated events.

The invention also provides a method of inducing growth, including proliferation and/or differentiation, of hematopoietic stem cells. Hematopoietic stem cells are self-renewing cells that can differentiate into mature blood cells of all lineages. A key role for Wnt signaling in the growth and differentiation of hematopoietic stem cells has recently been discovered (Reya et al. Nature 423: 409-414 (2003). As disclosed herein, soluble Ryk polypeptides can enhance the growth inducing effects of Wnt on hematopoietic stem cells. Hematopoietic stem cell growth can be induced by contacting the hematopoietic stem cell with a Wnt polypeptide and a soluble Ryk polypeptide. Any Wnt polypeptide as disclosed herein can be used, including, for example, a Wnt3a, Wnt1, or Wnt4 polypeptide, or a functional fragment thereof. Other Wnt polypeptides (e.g., Wnt5) can also be used according to the inventive methods. Soluble Ryk polypeptides suitable for inducing growth of hematopoietic stem cells include any soluble Ryk that comprises an extracellular domain of a Ryk polypeptide.

The invention further provides to a composition that contains a soluble Ryk polypeptide and/or a polynucleotide encoding a soluble Ryk polypeptide. In addition, the composition can further include a Wnt polypeptide, such as Wnt 3a (e.g., SEQ ID NO:2, SEQ ID NO:4), Wnt1 (e.g., SEQ ID NO:6, SEQ ID NO:8), and Wnt4 (e.g., SEQ ID NO:10, SEQ ID NO:12), and/or a polynucleotide encoding the Wnt polypeptide. In one aspect, a composition of the invention can be formulated for administration to a subject (e.g., human subject), for example, to stimulate neurite outgrowth, to induce the growth and/or differentiation of cells such as neuronal cells, neuronal precursor cells, or hematopoietic cells; or to reduce or inhibit the growth of cells exhibiting unregulated growth (e.g., cancer cells).

A composition of the invention can be prepared for administration to a subject by mixing the Wnt and/or soluble Ryk polypeptides (and/or encoding polynucleotides) with a physiologically acceptable carrier, which is nontoxic in the amount employed. Preparation of such a composition can include combining the Wnt and or soluble Ryk components with saline, buffers, antioxidants such as ascorbic acid, low molecular weight (less than about 10 residues) polypeptides, proteins, amino acids, carbohydrates including glucose or dextrans, or chelating agents such as EDTA, glutathione and/or other stabilizers and excipients of interest. In this respect, it should be noted that a composition of the invention can include one or more other agents that can provide, for example, a therapeutic advantage to a subject to be treated. As such, the composition can contain a diagnostic reagent, nutritional substance, toxin, a therapeutic agent, for example, a cancer chemotherapeutic agent, and, where the cell is a hematopoietic stem cell, the composition can contain a growth and or differentiation factor that, for example, directs differentiation along a desired pathway (e.g., GM-CSF). Such compositions can be maintained, for example, as a suspension or an emulsion, or can be lyophilized, then formulated as desired under conditions such that they are suitably prepared for use in the desired application. As such, the compositions are useful as medicaments for use in treating a disorder or for a purpose as disclosed herein.

A physiologically acceptable carrier can be any material that, when combined with an a Wnt and/or Ryk polypeptide and/or encoding polynucleotide allows the ingredient to retain the desired biological activity. Examples of such carriers include any of the standard physiologically acceptable carriers such as a phosphate buffered saline solution, water, emulsions such as oil/water emulsion, and various types of wetting agents. Diluents for aerosol or parenteral administration include phosphate buffered saline or normal (0.9%) saline. Compositions comprising such carriers are formulated by well known conventional methods (see, for example, Remington's Pharmaceutical Sciences, Chapter 43, 14th Ed., Mack Publishing Co., Easton Pa. 18042, USA). Further, the carrier can be an aqueous solution such as physiologically buffered saline or other solvent or vehicle such as a glycol, glycerol, an oil such as olive oil or an injectable organic esters. A carrier also can include a physiologically acceptable compound that acts, for example, to stabilize the peptide or encoding polynucleotide or to increase its absorption. Physiologically acceptable compounds include, for example, carbohydrates, such as glucose, sucrose or dextrans, antioxidants, such as ascorbic acid or glutathione, chelating agents, low molecular weight proteins or other stabilizers or excipients.

It will be recognized to the skilled clinician that the choice of a carrier, including a physiologically acceptable compound, depends, for example, on the manner in which the peptide or encoding polynucleotide is to be administered, as well as on the route of administration of the composition. A composition can be administered, for example, intramuscularly, intradermally, or subcutaneously, and also can be administered parenterally such as intravenously, and can be administered by injection, intubation, or other such method known in the art. A composition comprising a peptide or polynucleotide also can be incorporated within an encapsulating material such as into an oil-in-water emulsion, a microemulsion, micelle, mixed micelle, liposome, microsphere or other polymer matrix (see, for example, Gregoriadis, Liposome Technology, Vol. 1 (CRC Press, Boca Raton, Fla. 1984); Fraley, et al., Trends Biochem. Sci., 6:77, 1981, each of which is incorporated herein by reference). Liposomes, for example, which consist of phospholipids or other lipids, are nontoxic, physiologically acceptable and metabolizable carriers that are relatively simple to make and administer. “Stealth” liposomes (see, for example, U.S. Pat. Nos. 5,882,679; 5,395,619; and 5,225,212, each of which is incorporated herein by reference) are an example of such encapsulating material. Cationic liposomes, for example, also can be modified with specific receptors or ligands (Morishita et al., J. Clin. Invest., 91:2580-2585, 1993, which is incorporated herein by reference). In addition, a polynucleotide agent can be introduced into a cell using, for example, adenovirus-polylysine DNA complexes (see, for example, Michael et al., J. Biol. Chem. 268:6866-6869, 1993, which is incorporated herein by reference).

The present invention further relates to kits, which can include, for example, a soluble Ryk polypeptide and/or a Wnt polypeptide, and/or a polynucleotide encoding soluble Ryk and/or Wnt. A kit of the invention can additionally contain a container means, generally a vessel, glass vial or jar, or plastic pack. In one embodiment, the container is a single vessel that contains a soluble Ryk polypeptide and a Wnt polypeptide. In another embodiment, the kit includes a first and a second container, wherein one container contains a soluble Ryk polypeptide and the other container contains a Wnt polypeptide.

The invention further provides a method of modulating an effect of Wnt on a cell. The method includes contacting the cell with a soluble Ryk polypeptide that selectively binds to at least one of Wnt and Frizzled, whereby selective binding of a soluble Ryk polypeptide affects Ryk mediated Wnt signal transduction in the cell. The soluble Ryk polypeptide, such as a Ryk extracellular domain, can affect Ryk mediated signal transduction, for example, by specifically interacting with Wnt and Frizzled, and not Disheveled. Various types of cells as disclosed herein are amenable to treatment according to the present method, including, for example, neuronal, neuronal precursor, and cancer cells.

The invention also provides screening assays for identifying agents useful for practicing methods of the invention. In one embodiment, the invention provides a method of identifying an agent that modulates Ryk mediated signal transduction, for example, by contacting a sample having Ryk and Frizzled with a test agent, under conditions suitable for binding of Wnt to Ryk and Frizzled, and detecting a change in Ryk mediated signal transduction due to selective binding of the agent to Ryk and Frizzled. The sample can be a cell expressing Ryk and Frizzled, such as a neuronal, neuronal precursor, or cancer cell. Ryk and Frizzled can be expressed endogenously in the cell or experimentally introduced, for example, by transfection of the cell with an expression vector including Ryk or Frizzled. The sample can further include a Wnt polypeptide. The present invention further relates to agents identified by the inventive methods as capable of modulating Ryk activity.

In another embodiment, the screening assay provides a method of identifying an agent that modulates a specific interaction of Ryk and Frizzled. Such a method includes contacting a sample having Ryk and Frizzled with a test agent, under conditions suitable for formation of a Ryk/Frizzled complex suitable for Ryk mediated signal transduction, and detecting a change in the complex in the presence of the test agent as compared to complex formation in the absence of the test agent. In still another embodiment, the screening assay provides a method of identifying an agent that modulates a specific interaction of Ryk and Disheveled. The methods include contacting a sample having Ryk and Disheveled with a test agent, under conditions suitable for formation of a Ryk/Disheveled complex suitable for Ryk mediated signal transduction, and detecting a change in formation of the complex in the presence of the test agent as compared to complex formation in the absence of the test agent.

A change in Ryk mediated signal transduction can be detected in a variety of ways, including, for example, by detecting changes in downstream components of Ryk mediated signal transduction. A change in Ryk mediated signal transduction can be detected, for example, by detecting a change in TCF activation in the presence of the test agent as compared to TCF activation in the absence of the test agent. As discussed above, assays useful for detecting protein complex formation also can be used to detect the efficacy of a test agent examined according to a screening assay of the invention.

A test agent useful in the screening can be any molecule that is to be examined for the ability to modulate Ryk activity, including test agents to be examined for agonist activity, antagonist activity, partial agonist activity, and the like. As used herein, the term “agonist” refers to an agent that can specifically bind to a polypeptide involved in Ryk mediated signal transduction and increase Ryk mediated signal transduction activity. The term “partial agonist” is used herein to refer to an agent that has an effect similar to, but less than, that of an agonist. The term “antagonist” is used herein to refer to an that can bind, competitively or non-competitively, to a polypeptide involved in Ryk mediated signal transduction at substantially the same site as an agonist, but that does not increase Ryk mediated signal transduction activity It should be recognized that an agent identified according to a method of the invention acts by contacting Ryk, thereby effecting its activity, or by modulating an interaction of Ryk with a Ryk signal transduction pathway component (e.g., Wnt, Frizzled, and/or Dishevelled).

A test agent can be any molecule of interest, including, for example, a peptide, polynucleotide, peptidomimetic, or small organic molecule. Further, the test agent can be one of a library of test agents, for example, a combinatorial library of test agents, which can be a random library, a biased library, or a variegated library, which can comprise test agents based on a general structure of known Ryk, Wnt, Frizzled, or Disheveled proteins. Polynucleotides, for example, are known to specifically interact with proteins and, therefore, can be useful as test agents to be screened for the ability to selectively bind to a polypeptide involved in Ryk mediated signal transduction.

A polynucleotide agent can act, for example, by binding Ryk and reducing or inhibiting the ability of Wnt to bind Ryk, thus acting as an antagonist, or can act to induce the Ryk signal transduction pathway Polynucleotides that specifically bind to polypeptides are well known in the art, and polynucleotides that selectively bind to Ryk, for example, can be identified using well known methods (see, e.g., O'Connell et al., supra, 1996; Tuerk and Gold, supra, 1990; Gold et al., supra,.1995).

A peptide also can be useful as an agent that selectively binds a polypeptide involved in Ryk mediated signal transduction. The term “peptide” is used broadly herein to mean two or more amino acids linked by a peptide bond. Generally, a peptide useful in a method of the invention contains at least about two, three, four, five, or six amino acids, and can contain about ten, fifteen, twenty or more amino acids. As such, it should be recognized that the term “peptide” is not used herein to suggest a particular size or number of amino acids comprising the molecule, and that a peptide of the invention can contain up to several amino acid residues or more. Generally, however, smaller peptides are preferred where an identified agent is to be further examined, for example, for use as a drug for treating a subject. A peptide test agent can be prepared, for example, by a method of chemical synthesis, or can be expressed from a polynucleotide using recombinant DNA methodology. Where chemically synthesized, peptides containing one or more D-amino acids, or one or more amino acid analogs, for example, an amino acid that has been derivatized or otherwise modified at its reactive side chain, or in which one or more bonds linking the amino acids or amino acid analogs is modified, can be prepared. In addition, a reactive group at the amino terminus or the carboxy terminus or both can be modified. Such peptides can be modified, for example, to have improved stability to a protease, an oxidizing agent or other reactive material the peptide may encounter in a biological environment, and, therefore, can be particularly useful in performing a method of the invention. A peptide also can be modified by glycosylation, which can be effected by linking a carbohydrate moiety to a reactive side chain of an amino acid of the peptide or by including one or a few additional amino acids at the N-terminus or C-terminus of the peptide and linking the carbohydrate moiety to the additional amino acid. The linkage can be any linkage commonly found in a glycoprotein, for example, an N-linked or O-linked carbohydrate to an asparagine residue or a serine residue, respectively, or can be any other linkage that conveniently can be effected. Of course, the peptides can be modified to have decreased stability in a biological environment such that the period of time the peptide is active in the environment is reduced.

An agent or ligand that selectively binds to a polypeptide involved in Ryk mediated signal transduction can also be identified using methods of molecular modeling. Modeling systems useful for the purposes disclosed herein can be based on structural information obtained, for example, by crystallographic analysis or nuclear magnetic resonance analysis, or on primary sequence information (see, for example, Dunbrack et al., “Meeting review: the Second meeting on the Critical Assessment of Techniques for Protein Structure Prediction (CASP2) (Asilomar, California, Dec. 13-16, 1996). Fold Des. 2(2): R27-42, (1997); Fischer and Eisenberg, Protein Sci. 5:947-55, 1996; (see, also, U.S. Pat. No. 5,436,850); Havel, Prog. Biophys. Mol. Biol. 56:43-78, 1991; Lichtarge et al., J Mol. Biol. 274:325-37, 1997; Matsumoto et al., J. Biol. Chem. 270:19524-31, 1995; Sali et al., J. Biol. Chem. 268:9023-34, 1993; Sali, Molec. Med. Today 1:270-7, 1995a; Sali, Curr. Opin. Biotechnol. 6:437-51, 1995b; Sali et al., Proteins 23: 318-26, 1995c; Sali, Nature Struct. Biol. 5:1029-1032, 1998; U.S. Pat. No. 5,933,819; U.S. Pat. No. 5,265,030, each of which is incorporated herein by reference).

The crystal structure coordinates of a polypeptide involved in Ryk mediated signal transduction can be used to design compounds that bind to the protein and alter its physical or physiological properties in a variety of ways. The structure coordinates of the protein can also be used to computationally screen small molecule data bases for agents that bind to the polypeptide to develop modulating or binding agents, which can act as agonists or antagonists of Ryk or Wnt activity. Such agents can be identified by computer fitting kinetic data using standard equations (see, for example, Segel, “Enzyme Kinetics” (J. Wiley & Sons 1975), which is incorporated herein by reference).

Methods of using crystal structure data to design inhibitors or binding agents are known in the art. For example, Ryk coordinates can be superimposed onto other available coordinates of similar receptors, including receptors having a bound inhibitor, to provide an approximation of the way the inhibitor interacts with the receptor. Computer programs employed in the practice of rational drug design also can be used to identify compounds that reproduce interaction characteristics similar to those found, for example, between a Ryk and ligand such as a Wnt polypeptide or fragment thereof. Detailed knowledge of the nature of the specific interactions allows for the modification of compounds to alter or improve solubility, pharmacokinetics, and the like, without affecting binding activity.

Computer programs for carrying out the activities necessary to design agents using crystal structure information are well known. Examples of such programs include, Catalyst Databases™—an information retrieval program accessing chemical databases such as BioByte Master File, Derwent WDI and ACD; Catalyst/HYPO™—generates models of compounds and hypotheses to explain variations of activity with the structure of drug candidates; Ludi™—fits molecules into the active site of a protein by identifying and matching complementary polar and hydrophobic groups; and Leapfrog™—“grows” new ligands using a genetic algorithm with parameters under the control of the user.

As disclosed herein, the methods of the invention provide the advantage that they can be adapted to high throughput analysis and, therefore, can be used to screen combinatorial libraries of test agents in order to identify those agents that can selectively bind to a polypeptide involved in Ryk mediated signal transduction. Methods for preparing a combinatorial library of molecules that can be tested for a desired activity are well known in the art and include, for example, methods of making a phage display library of peptides, which can be constrained peptides (see, for example, U.S. Pat. No. 5,622,699; U.S. Pat. No.5,206,347; Scott and Smith, Science 249:386-390, 1992; Markland et al., Gene 109:13-19, 1991; each of which is incorporated herein by reference); a peptide library (U.S. Pat. No. 5,264,563, which is incorporated herein by reference); a peptidomimetic library (Blondelle et al., Trends Anal. Chem. 14:83-92, 1995; a nucleic acid library (O'Connell et al., supra, 1996; Tuerk and Gold, supra, 1990; Gold et al., supra, 1995); an oligosaccharide library (York et al., Carb. Res., 285:99-128, 1996; Liang et al., Science, 274:1520-1522, 1996; Ding et al., Adv. Expt. Med. Biol. 376:261-269,1995; each of which is incorporated herein by reference); a lipoprotein library (de Kruif et al., FEBS Lett. 399:232-236, 1996, which is incorporated herein by reference); a glycoprotein or glycolipid library (Karaoglu et al., J. Cell Biol. 130:567-577, 1995, which is incorporated herein by reference); or a chemical library containing, for example, drugs or other pharmaceutical agents (Gordon et al., J. Med. Chem. 37:1385-1401, 1994; Ecker and Crooke, BioTechnology 13:351-360, 1995; each of which is incorporated herein by reference). Polynucleotides can be particularly useful as agents that can modulate a specific interaction of an agent or ligand and a polypeptide involved in Ryk mediated signal transduction because nucleic acid molecules having binding specificity for cellular targets, including cellular polypeptides, exist naturally, and because synthetic molecules having such specificity can be readily prepared and identified (see, for example, U.S. Pat. No. 5,750,342, which is incorporated herein by reference).

In performing a screening assay of the invention in a high throughput format, isolated Ryk or soluble Ryk polypeptide, cell membranes containing Ryk, or intact cells expressing Ryk can be used. An advantage of using intact cells is that the method can be used, for example, to identify an agent that selectively binds Ryk or other polypeptides involved in Ryk mediated signal transduction in particular cells or cell types. For example, a plurality of cells from a subject can be arranged in an array, which can be an addressable array, on a solid support such as a microchip, on a glass slide, on a bead, or in a well, and the cells can be contacted with different test agents to identify one or more agents having desirable characteristics, including, for example, in addition to selectively binding to the polypeptide, minimal or no toxicity to the cell, desirable solubility characteristics, and the like. An additional advantage of arranging the samples in an array, particularly an addressable array, is that an automated system can be used for adding or removing reagents from one or more of the samples at various times, or for adding different reagents to particular samples. In addition to the convenience of examining multiple samples at the same time, such high throughput assays provide a means for examining duplicate, triplicate, or more aliquots of a single sample, thus increasing the validity of the results obtained, and for examining control samples under the same conditions as the test samples, thus providing an internal standard for comparing results from different assays.

In one embodiment, a plurality of test agents (e.g., a combinatorial library of test agents) is examined for selective binding to a polypeptide involved in Ryk mediated signal transduction. Advantages of performing the present methods in a high throughput format include, for example, that duplicates, triplicates, or more of an assay can be performed, whereby statistically significant results can be obtained; and that one or more (positive and/or negative) controls can be performed in parallel, thus providing a means to obtain standardized results (e.g., among samples performed at different times or under different conditions).

The following examples are intended to illustrate but not limit the invention.

### EXAMPLE 1

### Ryk is a Co-receptor for Wnt and Required for Wnt Dependent Stimulation of Neurite Outgrowth

This example demonstrates that Ryk is involved in the Wnt mediated signal transduction pathway and is involved in neurite outgrowth of neuronal and neuronal precursor cells.

Transient transfection, coimmunoprecipitation and Western blotting [01081 293T cells were grown in DMEM supplemented with 10% FBS, 100 μg/ml of penicillin and streptomycin, and 2 mM glutamine in a 37° C. incubator with 5% humidified CO₂. Twenty-four hours before transfection, 4 million 293T cells were seeded in 10 cm dishes. The cells were transfected with plasmid DNA using the calcium phosphate precipitation method. For Wnt/Ryk interaction, a total of 16 μg DNA was transfected, including 8 μg of HA-tagged Wnt-1 or Wnt 3a, and 8 tg myc-tagged Ryk or its mutants.

For interaction of Ryk and Dishevelled, 8 μg of EBG-Ryk 314-C or EBG-Ryk APDZ was transfected with 8 μg of plasmid for flag-tagged Dishevelled (APDZ). Forty-eight hours post-transfection, cells were lysed in 1 ml ice-cold kinase lysis buffer (25 ml Tris PH 7.4, 150 mM NaCl, 5 mM EDTA, 1% TRITON X-100 detergent, 10 mM sodium pyrophosphate, 10 mM β-glycerophosphate, 1 mM sodium orthovanadate, 10% glycerol and appropriate amount of protease inhibitor mix (Roche)). Monoclonal antibodies or affinity purified polyclonal antibodies (1 μg) were incubated with 200 μl cell lysate for 2 hours at 4° C. and precipitated with 10 μl protein G-agarose (Pierce). GST-Ryk was pulled down directly by glutathione-agarose beads (Amersham Biosciences). Immunoprecipitates were washed extensively for 4-5 times before SDS-PAGE analysis and immunoblotting. The primary antibodies used were anti-HA (1:200, Santa Cruz), anti-myc (1:200, Santa Cruz), and anti-flag (1:2000, Sigma). Anti-Dishevelled antibodies are a mixture of Dishevelled-1, 2 and 3 from Santa Cruz. Mouse polyclonal antiserum was generated using GST fusion protein of Ryk amino acid 236 to the C-terminus. The secondary antibodies were HRP-conjugated goat anti-mouse and goat anti-rabbit (1:10,000, Pierce).

Design of siRNA Constructs in pSUPER Vector and Lentiviral Vectors and Preparation of Lentivirus

Ryk siRNAs were designed and cloned into the pSUPER vector as described (Brummelkamp et al., 2002). siRNA-1 targets human Ryk 341 to 360. The two oligos used were:

| | |
|---|---|
| GATCCCCGTCCAGGTTGAATATAAGttcaagagaCT | (SEQ ID NO:17) |
| TATATTCAACCTTGGACTTTTTGGAAA; | |
| and | |
| AGCTTTTCCAAAAAGTCCAAGGTTGAATATAAGtct | (SEQ ID NO:18) |
| cttgaaCTTATATTCAACCTTGGACGGG. | |

siRNA-2 targets human Ryk 1659-1678. The sequences of the two oligos were:

| | |
|---|---|
| GATCCCCGATGGTTACCGAATAGCCCttcaagagaG | (SEQ ID NO:19) |
| GGCTATTCGGTAACCATCTTTTTGGAAA; | |
| and | |
| AGCTTTTCCAAAAAGATGGTTACCGAATAGCCCtct | (SEQ ID NO:20) |
| cttgaaGGGCTATTCGGTAACCATCGGG. | |

The siRNA oligos targeting Dishevelled-2 were as follows:

| | |
|---|---|
| GATCCCCCATGGAGAAGTACAACTTCttcaagagaG | (SEQ ID NO:21) |
| AAGTTGTACTTCTCCATGTTTTTGGAAA; | |
| and | |
| AGCTTTTCCAAAAACATGGAGAAGTACAACTTCtct | (SEQ ID NO:22) |
| cttgaaGAAGTTGTACTTCTCCATGGGG. | |

The siRNA oligos targeting Dishevelled-3 were as follows:

| | |
|---|---|
| GATCCCCGTTCTTCTTCAAGTCTATGttcaagagaC | (SEQ ID NO:23) |
| ATAGACTTGAAGAAGAACTTTTTGGAA; | |
| and | |
| AGCTTTTCCAAAAAGTTCTTCTTCAAGTCTATGtct | (SEQ ID NO:24) |
| cttgaaCATAGACTTGAAGAAGAACGGG. | |

In bold are regions identical to both human and mouse Ryk genes. Therefore, these siRNAs can be used in both human and mouse cells to target endogenous Ryk MRNA. Each pair of oligos was annealed at 20 μM in annealing buffer (100 mM potassium acetate, 30 mM Hepes-KOH, PH 7.4, 2 mM magnesium acetate) at 95° C. for 4 minutes, followed by incubation at 70° C. for 10 minutes and slow cooling to room temperature. Forty picomoles of annealed oligos were phosphorylated by T4 polynucleotide kinase before they were ligated into pSUPER vector digested by Bgl II and Hind III. To put siRNA constructs into lentiviral vectors, siRNA together with human HI promoter was digested with Sma I and Hinc II and ligated into pFUGW digested with Pac I followed by blunting using T4 DNA polymerase. The orientations of the fragments were confirmed by Cla I and Eco RI digestion. Lentivirus expressing siRNA were generated using retroviral vectors and a previously described packaging system (Lois et al., 2002). Concentrated lentivirus was titered using 293T cells to test GFP expression.

Real Time PCR

RNA from 293T cells, Ryk siRNA cells and mouse brains were extracted using tri-reagents (Molecular Research Center). First strand cDNA was synthesized using TAQMAN reverse transcription reagents (Applied Biosystems). The final concentration of the reaction was: 1× TAQMAN RT buffer, 5.5 mM MgCl₂, 500 μM of dNTPs, 2.5 μM of random hexamer, 0.4 u/μl of RNase inhibitor, 1.25 u/μl of MULTOSCRIBE reverse transcriptase and 10-100 ng of total RNA. The thermal cycling parameter of the RT reaction was 25° C. for 10 minutes, 48° C. for 30 minutes, and 95° C. for 5 minutes. The real time PCR was performed using the ABI5700 Real Time PCR Instrument. The reaction included 1× SYBR Green PCR Master Mix (Applied Biosystems), forward and reverse primer (0.5 μM each) and the appropriate amount of cDNA. The thermal cycling parameter was 50° C. for 2 minutes, 95° C. for 10 minutes, and 40 cycles of melting, annealing and extension. The melting condition was 95° C. for 15 seconds, annealing and extension was at 60° C. for 1 minute. Results are analyzed according to the manufacturer's instructions.

The primers for real time PCR were designed using Primer Express 1.5 software. The primers were designed to be around 160 bp with a Tm of 58-60° C.

Oligos used for amplification of human Ryk genes were:

| | |
|---|---|
| hryk-F2: | |
| AGGTGACAATGATGCTCACTGAA; | (SEQ ID NO:25) |
| hryk-R2: | |
| TGTGATGAAGACCTCGCAGCT; | (SEQ ID NO:26) |
| hryk-F3: | |
| CAGGTGACAATGATGCTCACTGA; | (SEQ ID NO:27) |
| and | |
| hryk-R3: | |
| GTGATGAAGACCTCGCAGCTTA. | (SEQ ID NO:28) |

Oligos used for human GAPDH were:

| | |
|---|---|
| GAPDH-F: | |
| GGTGGTCTCCTCTGACTTCAACA; | (SEQ ID NO:29) |
| and | |
| GALPDH-R: | |
| GCGTCAAAGGTGGAGGAGTG. | (SEQ ID NO:30) |

Northern Blot Analysis

The Northern blotting was performed as described (Tanaka et al., 1997). Radioactive-labeled anti-sense probes were used for hybridization. Antisense RNA probes were synthesized from pBS KSII Ryk236-C and pTri-GAPDG-mouse (Ambion) using T7 NA polymerase (Promega).

Luciferase Reporter Assay

293T cells were plated at 10⁵ cells per well in 24 well dishes 24 hours before transfection. The plasmids used in the transfection included 2 ng of pCSK-lacZ, 20 ng of topflash TCF-luc, and 350 ng of other DNA. Fopflash, which has a mutation in the TCF binding site was used as a control in some cases. The medium was changed 24 hours following transfection. 48 hours post-transfection, the cells were lysed in 100 μl of reporter lysis buffer (Promega). Cells were collected and spun at 13,000 rpm for 5 minutes. Twenty μl of supernatant were used to measure luciferase activity using the luciferase assay system (Promega) and a luminometer (Optocomp I, MGM instruments). Thirty μl of supernatant were used to measure the β-galactosidase activity using the chemiluminescent β-gal reporter gene assay (Roche) according to manufacturer's instructions. β-gal activity was used to normalize the amount of cell lysate.

Generation of Ryk siRNA Transgenic Mice Using Lentivirus

Transgenic mice expressing Ryk siRNA was generated as described (Lois et al., 2002). Approximately 10 to 100 μl of concentrated virus at 106 i.u./μl were injected into the perivitelline space of single-cell mouse embryos. Around 40 embryos were implanted into 2 timed pseudo pregnant female mice and carried to term. Genomic DNA from tails of transgenic mice was subjected to Southern blotting using a GFP-WRE DNA fragment as a probe. The mice were also tested for GFP expression by fluorescent microcopy of their tails. Transgenic mice were crossed with C57BL6 and mice carrying single copy transgenes were selected for experiments.

DRG Explants Collagen Gel Assay

E13 embryos from both wild type and Ryk siRNA mice were collected and washed with ice cold PBS. Dorsal root ganglia were isolated and incubated in LI 5 medium on ice. Ten μl of 10× DMEM/F12 were mixed with 90 μl of collagen gel (BD Biosciences) and put on ice. Ten μl of collagen gel mix were put on the surface of a small culture dish and placed at room temperature until the gel solidified. DRG explants were placed on top of this surface and another 20 μl of gel mix was added and incubated at 37° C. for 10 minutes. Two ml DMEM/F12 medium supplemented with Wnt3a conditioned medium or control conditioned medium were added. The explants were cultured between 24 to 72 hours before they were fixed for immunostaining.

Neurite outgrowth induced by concentrated Wnt3a or NGF is semi-quantified using IMAGEQUANT software. Briefly, the picture of neurite was first converted to grayscale. Background and explant core signal were subtracted so that only signals for neurite would be quantified and compared. With dilute Wnt3a, when the neurite number is low, the length of neurite is compared in arbitrary units.

Whole Mount Immunostaining of Mouse Embryos and DRG Explants in Collagen Gel.

The mouse embryos and DRG explants from the appropriate stage were fixed in 4% paraformaldehyde overnight. The tissues were then washed with PBS twice and then dehydrated serially in 25%, 50%, 75% and 100% methanol and stored in methanol overnight or longer. Prior to antibody staining, tissues were first treated with 0.3% H₂O₂ for half an hour followed by rehydration serially with 75%, 50%, 25% methanol, PBS and PBT (PBS+1% TWEEN detergent) twice, 5 minutes each. The tissues were blocked in PBS with 10% heat inactivated goat serum for 2 hours. Primary antibody 2H3 (hybridoma cell bank) at 1:50 dilution in PBS was added for incubation overnight followed by 6 washes, 30 min each with PBS/2% goat serum. The HRP-conjugated goat anti-mouse IgG1 antibody (Southland Biotechnology, 1:300) was incubated with the tissues for 2 hours followed by 6 washes of PBS/2% goat heat inactivated serum, 30 minutes each. The color reaction was developed using DAB staining.

Ryk binds to Wnt-1 and Wnt-3A Through the Extracellular WIF Domain

The extracellular domain of Ryk is homologous to Wnt inhibitory factor (WIF) (Hsieh et al., 1999; Patthy, 2000), suggesting that Wnt may be a ligand for Ryk. To test this hypothesis, an examination of whether Ryk can bind to Wnt proteins was performed. Myc-tagged Ryk, myc-tagged Ryk with its extracellular domain deleted (RykAEx), and myc-tagged Ryk with its intracellular domain deleted (RykAln) were cotransfected into 293T cells with either HA-tagged Wnt-1 or Wnt-3a. Cell lysates from the transfected cells were subjected to immunoprecipitation with anti-myc antibody followed by Western blotting with an anti-HA antibody. Both Wnt-1 and Wnt-3a bound Ryk. The extracellular WIF domain was required for both interactions because the RykAEx mutant did not coimmunoprecipitate with Wnt-1 or Wnt-3a. The intracellular domain of Ryk did not contribute to the Ryk/Wnt interaction because RykAIn bound effectively to both Wnt-1 and Wnt-3a. Thus, Ryk had the properties of a receptor for Wnt proteins.

Ryk Protein is Required for TCF-driven Transcription.

Activation of the canonical Wnt signaling pathway requires β-catenin stabilization and its association with TCF to activate transcriptional targets (Moon et al., 2002; Wodarz and Nusse, 1998). To determine if Ryk binding by Wnt leads to activation of the canonical Wnt pathway, a TCF-mediated luciferase reporter assay was utilized. 293T cells were co-transfected with a TCF-luciferase reporter construct and a DNA construct expressing Ryk. Forty-eight hours post-transfection, cells were treated with Wnt 3a-conditioned medium for 6 hours and lysed to determine luciferase activity. Ryk activated the TCF-luciferase reporter 1.5-fold while Wnt3a activated the TCF-luciferase reporter 2-fold to 3-fold (FIG. 1). However, the TCF-luciferase reporter was activated 5-fold in the cells transfected with Ryk and treated with Wnt3a conditioned medium. A control mutant TCF-Luciferase reporter (Fopflash) was used and no activation was found. These results suggested that Wnt3a and Ryk might function cooperatively in the activation of the TCF-luciferase reporter and supported the notion that Ryk is a receptor for Wnt.

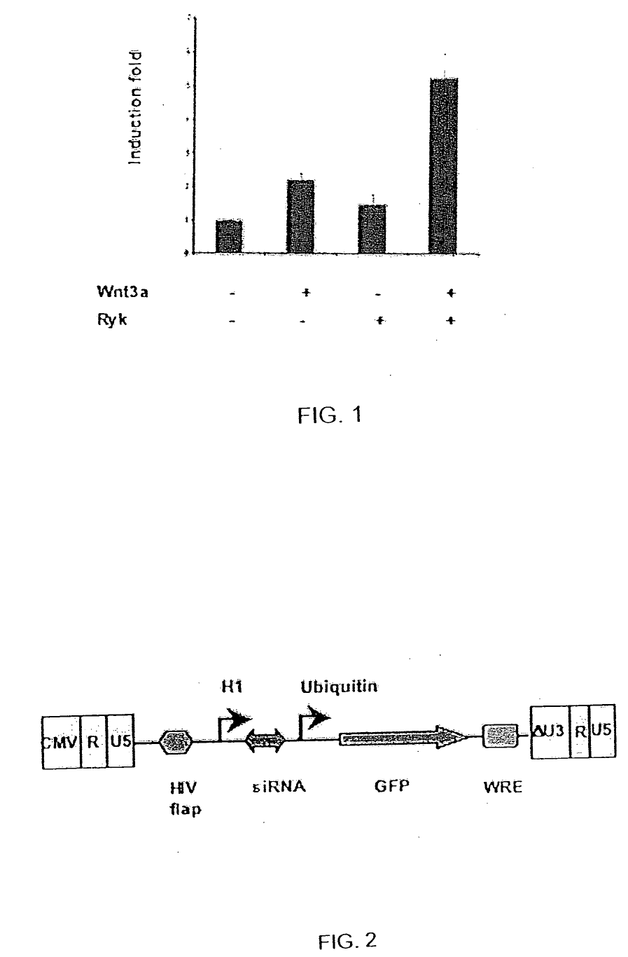

To definitively ask whether Ryk plays an essential role in the Wnt signaling pathway, siRNA technology was used to knock down the expression of the endogenous Ryk gene. Four hairpin double-strand DNA sequences to target Ryk were designed and tested for their ability to reduce Ryk expression (Brummelkamp et al., 2002). The strongest Ryk siRNAs, driven by an H1 promoter, were inserted into the lentiviral vector FUGW as illustrated (FIG. 2) (Lois et al., 2002). VSVG pseudotyped lentivirus was generated. A stable mouse L cell line expressing myc-tagged Ryk was infected with either siRNA or control virus. When 100% of cells were infected, myc-tagged Ryk expression was completely inhibited.

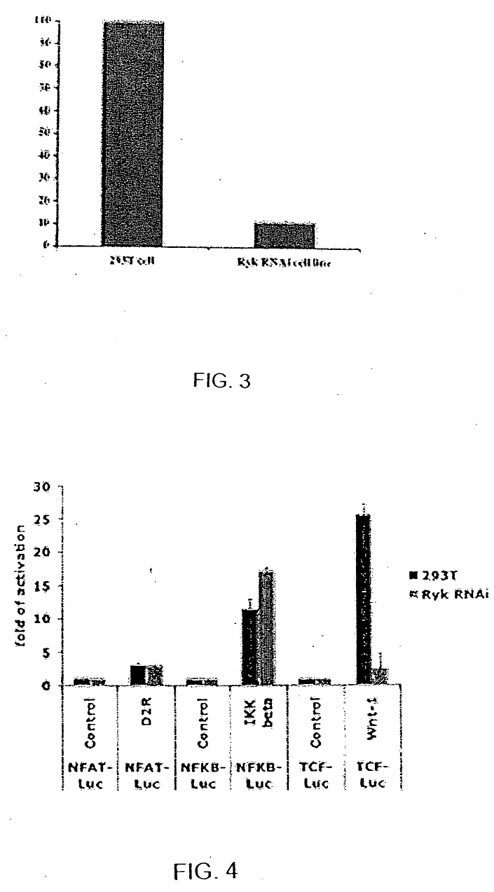

To test whether Ryk plays a role in Wnt signal transduction, 293T cell lines expressing Ryk siRNA were generated. Endogenous Ryk mRNA levels were determined by real time PCR. Messenger RNA of human GAPDH was used as an internal control. Compared with the control, Ryk MRNA from the Ryk siRNA cell line was inhibited by 88% (FIG. 3).

Signal transduction in these cells was examined using a luciferase reporter assay. In wild type cells, the TCF-luciferase reporter was activated about 25-fold after Wnt stimulation, while in Ryk siRNA-containing cells, TCF-driven transcription induced by Wnt-1 was greatly inhibited (FIG. 4). This suggests that Ryk is required for Wnt-induced, TCF-driven transactivation. As controls, the activation of an NF-kappaB-luciferase reporter by IKK and an NFAT-luciferase reporter by the Dopamine receptor 2 (D2R) were not inhibited in the Ryk siRNA-containing cells, demonstrating that the Ryk siRNA-mediated inhibition was specific to the TCF pathway. These results strongly support the hypothesis that Ryk is a functional receptor for Wnt.

Ryk Forms a Ternary Complex with Frizzled and Wnt.

Since Frizzled is the canonical receptor for Wnt (Bhanot et al., 1996), an investigated was performed to determine whether Ryk acts as a co-receptor with Frizzled. The extracellular cysteine rich domain (CRD) of Frizzled-8 was fused with the human IgG Fc fragment and was cotransfected into 293T cells with increasing amounts of myc-tagged Ryk extracellular domain with or without HA-tagged Wnt-1. As a negative control, the Fc fragment was also transfected into 293T cells. The Fc and Fc fusions were immunoprecipitated with protein A/G agarose. Associated proteins were determined by Western blotting using anti-HA and anti-myc antibodies. In the absence of Wnt-1, the CRD of Frizzled-8 binds strongly to the Ryk extracellular domain, while Fc alone does not. In the absence of Ryk, the Frizzled CRD binds to Wnt as well. Furthermore, overexpression of increasing amounts of Ryk does not inhibit the Wnt/Frizzled interaction, suggesting that Ryk may form a ternary co-receptor complex with Frizzled and Wnt.

Ryk Links Wnt to Dishevelled.

The present results establish that Ryk can be involved in the canonical pathway of Wnt signaling but it is not clear whether it involves Dishevelled, one of the key components of the Wnt pathway. First, an examination as to whether there was any interaction of endogenous Ryk and Dishevelled was performed. Both 293T cell lysate and mouse brain cell lysate were subjected to immunoprecipitation with mice polyclonal anti-Ryk serum followed by immunoblotting with anti-Dishevelled antibody. Dishevelled was found to coimmunoprecipitate with Ryk in both 293T cells and brain cells, suggesting that endogenous Ryk and Dishevelled associated with each other in vivo. It was reasoned that the binding was most likely mediated by the PDZ domain of Dishevelled and the c-terminal PDZ binding motif of Ryk. To test this hypothesis, the GST fusion protein of the Ryk intracellular domain and its PDZ binding domain deletion mutant were transfected with Dishevelled (ADIX) into 293T cells. The Dishevelled (ADIX) mutant was used because the DIX domain is associated with lipids and actin and caused a high background in coimmunoprecipitation experiments. Western blotting of the GST Ryk immunoprecipitate showed that the flag-tagged Dishevelled mutant associated with Ryk. This association was mediated by the PDZ binding motif of Ryk because its deletion abolished binding.

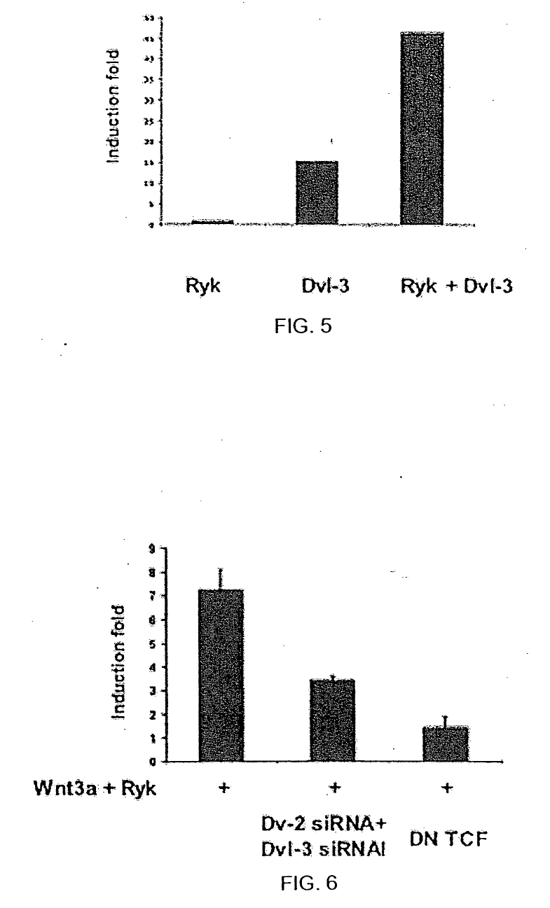

Whether Ryk and Dishevelled can synergize in activating the TCF pathway was examined. Overexpression of Dishevelled led to the activation of the TCF-luciferase reporter about 15-fold. Co-expression of Ryk further enhanced the activation to about 25-fold, supporting the hypothesis of a functional interaction between Ryk and Dishevelled (FIG. 5). Next, as investigation as to whether the activation of TCF-luciferase reporter induced by Wnt3a and Ryk is mediated by Dishevelled was performed. This was done by knocking down the Dishevelled expression by RNAi. In 293T cells Dishevelled-2 and Dishevelled-3 are expressed while Dishevelled-1 is not. Therefore, siRNA against Dishevelled 2 and 3 were used to inhibit the expression of endogenous Dishevelled in 293 cells. SiRNA for Dishevelled-2 blocked the expression of Dishevelled-2 specifically and had no effect on Dishevelled 3, while Dishevelled-3 siRNA only knocked down the expression of Dishevelled-3. Overexpression of siRNA against Dishevelled-2 and siRNA against Dishevelled-3 together blocked the TCF-Luciferase reporter activation in the cells transfected with Ryk gene and treated with Wnt3a conditioned medium, as did the dominant negative TCF-4 (FIG. 6). These results demonstrate that Ryk regulates the canonical TCF pathway by acting with Dishevelled protein. The interaction of Dishevelled and Ryk provides a novel link between Wnt and Dishevelled.

Generation of Ryk siRNA Mice

To address the roles of Ryk in in vivo neural development, transgenic mice expressing Ryk siRNA were generated by lentiviral infection of mouse one-cell stage embryos. These embryos were transferred to pseudopregnant recipient mice and those that came to term were examined further. Transgenesis was determined by fluorescent microscopy of mouse tails and FACS analysis of tail blood. Among 18 offspring, eight mice were GFP positive. Three of them were runted. The copy number of each transgene was determined by Southern blot analysis. Most transgenic lines had three to four copies of integrated lentiviral transgenes. The relative radiographic density of some transgene bands was weak, suggesting these transgenic lines might be mosaic. FACS analysis of tail blood further confirmed that the transgenic mice were mosaic, as less than 30% of white blood cells were GFP positive. Mice with multiple copies of the transgenes were mated to wild type C57BL6 mice to segregate non-mosaic, single transgene-containing lines. Offspring were analyzed for Ryk RNA levels using real time PCR in combination with Northern blotting. Northern blot analysis showed that the Ryk niRNA level in the brain from one line of Ryk siRNA mice was reduced 5- to 10-fold.

Many Ryk siRNA transgenic mice died after birth, as has been observed with Ryk knockout mice (Halford et al., 2000). Some of the surviving mice were runted. These were about half the size of their control siblings on Day 3 and 8. The difference in weight became less dramatic over time. These mice also displayed developmental defects, such as an unsteady gait.

Ryk siRNA Mice Have Defects in Axon Guidance

Drosophila derailed is involved in axon guidance as well as learning and memory (Callahan et al., 1995; Dura et al., 1995; Moreau-Fauvarque et al., 1998; Simon et al., 1998). Therefore the role of Ryk in axon guidance also was examined using Ryk siRNA transgenic mice. Neurafilament staining of E10 embryos showed that the majority of axons projected correctly in Ryk siRNA mice, compared to wild type mice. However, glossopharyngeal nerves and vagus nerves prematurely connected, and craniofacial motor neuron axons were less fasciculated in Ryk siRNA mice. In the E10.5 embryos of Ryk siRNA mice, the ophthalmic axons, instead of projecting to the anterior, wandered posterior and were less fasciculated. The projection and fasciculation of the DRG axons was normal.

Wnt3a Induces Neurite Outgrowth in DRG Neurons

In Drosophila, Ryk homolog, Derailed, is involved in the regulation of the guidance of anterior commissural axons (Bonkowsky et al., 1999; Yoshikawa et al., 2003). Moreover, Wnt has been shown to be involved in axon arborization in rats (Hall et al., 2000; Krylova et al., 2002; Lucas and Salinas, 1997). To establish a system for examining the role of Ryk in in vitro neuronal development, dorsal root ganglion (DRG) explants from rat E14 embryos were co-cultured with a mouse L cell line overexpressing Wnt. Sixteen hours afterwards, the explants were fixed and immunostained for synapsin expression, a marker for presynaptic terminals. Compared with the control cell line, Wnt3a, 4 and Wnt7b all induced a significantly greater expression of synapsin.

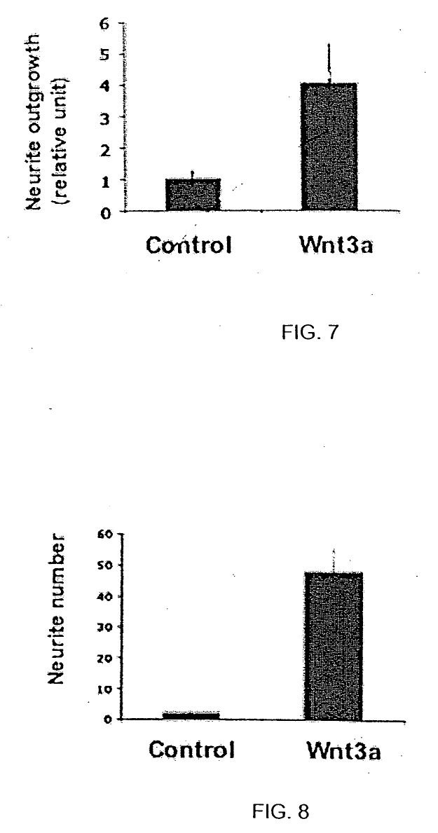

Not only was the synapsin signal significantly increased in the Wnt3a treated DRG explants, neurite numbers were also visibly increased as demonstrated by staining for GAP43 protein, suggesting that Wnt3a can induce neurite outgrowth. To further confirm the role of Wnt3a in neurite outgrowth, DRG explants from E13 mouse embryos were harvested, placed in a collagen gel, and incubated in DMEM/F12 growth medium supplemented with concentrated Wnt3a conditioned medium. The control explants were cultured in the same medium plus an addition of concentrated conditioned medium from normal L cells. Neurites were visualized using neurafilament antibody (2H3) after 24-48 hours of culture. The explants in the normal L cell conditioned medium had few neurites while the number and length of neurites in Wnt3a conditioned medium was dramatically increased (FIG. 7). These results support the role of Wnt in inducing neurite outgrowth. The dilute Wnt3a conditioned medium had a similar effect (FIG. 8).

It has been reported that Ryk is expressed in DRGs (Kamitori et al., 1999). However, the localization of Ryk in DRG neurons was not known. The anti-Ryk antiserum generated in mice was not sufficient; therefore, to detect the localization of Ryk in DRG neurons, dissociated DRG neurons were infected with lentivirus expressing a Ryk/GFP fusion. The expression of Ryk/GFP and GAP 43 was determined by immunohistochemistry. The overlay of Ryk/GFP and GAP 43 suggested that Ryk was localized not only in the cell body but also in growth cones, consistent with its roles in neurite outgrowth and axon guidance.

Ryk siRNA Mice Have Defects in Neurite Outgrowth in Response to Wnt3a Induction

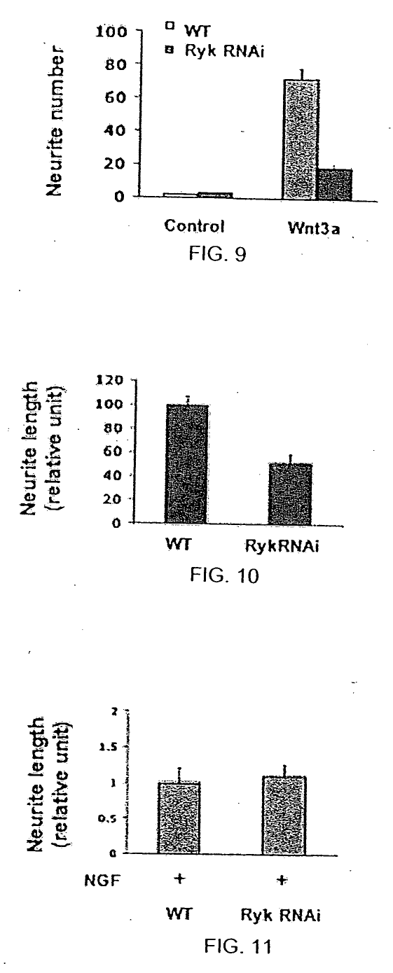

Although our results showed that Wnt3a induced neurite outgrowth in DRG explants, the DRG axon outgrowth looked normal in Ryk siRNA mice. Ryk mRNA level in the DRG explants isolated from Ryk siRNA mice was inhibited to 14% of the wild type control. To assess whether the DRG neurons in Ryk siRNA mouse had defects in neurite outgrowth in response to Wnt stimulation, DRG explants were isolated from E13 embryos of both wild type and Ryk siRNA mice, and cultured in a collagen gel with DMEM/F12 supplemented with unconcentrated Wnt3a conditioned medium. While numerous neurites projected from the wild type DRG explants, DRG explants from Ryk siRNA mice had fewer and shorter neurites emanating from them. The number of neurites decreased 4-fold (FIG. 9), while the average length of neurites was reduced by 2-fold (FIG. 10). As a control, neurite outgrowth from wild type and Ryk siRNA DRG was similar when the medium was supplemented with nerve growth factor (NGF) (FIG. 11), suggesting that Ryk is specifically involved in Wnt-induced neurite outgrowth. This may also explain why the neurite outgrowth of DRG is normal in Ryk siRNA mice since NGF and other growth factors might be also involved in inducing neurite outgrowth in vivo. The demonstration that Ryk is required for the Wnt3a-induced neurite outgrowth and the binding of Ryk and Wnt3a indicates that Ryk is a biological receptor of Wnt in vivo.

### EXAMPLE 2

### Ryk as a Target for Cancer Therapy

This Example demonstrates that Ryk is involved in the growth and proliferation of cancer cells.

Ryk is Highly Expressed in Cancer Cells. =P Ryk expression level was exceptionally high in the cancer cell. The total RNA from mouse brain and human cancer cell line 293T cells were extracted, then subject to Northern blotting using the radio-labeled antisense RNA as a probe. GAPDH, a housekeeping gene that is ubiquitously expressed in all mammalian cells, was used as an internal control for loading. The expression of Ryk in the 293 cells is over ten-fold more than the normal brain tissue (see Example 1).

Ryk Extra-cellular Domain Binds Wnt-1.

Myc tagged Ryk, an extracellular domain deletion mutant (RykAEx) or an intracellular domain deletion mutant (RykAln) was co-transfected into 293 cells with HA tagged Wnt-1. Cell lysates from the transfected cells were subjected to immunoprecipitation using anti-myc antibody and subsequent Western blotting using anti-HA antibody. Wnt-1 was shown to be associated with Ryk. Furthermore, the extracellular domain was required for the interaction while the intracellular domain was not (See above).

Inhibition of Ryk Expression Blocks the Activation of TCF Pathway Induced by Wnt-1.

293T cell lines expressing Ryk siRNA were generated. Endogenous Ryk mRNA levels were determined by real time PCR. Messenger RNA of human GAPDH was used as an internal control. Compared with the control, Ryk mRNA from the Ryk siRNA cell line was inhibited by 88% (FIG. 3).

Signal transduction in these cells was examined using a luciferase reporter assay. In wild type cells, the TCF-luciferase reporter was activated about 25-fold after Wnt stimulation, while in Ryk siRNA-containing cells, TCF-driven transcription induced by Wnt-1 was inhibited about 12-fold (FIG. 4). This suggests that Ryk is required for TCF-driven transactivation. As controls, the activation of an NF-kappa B-luciferase reporter by IKK and an NFAT-luciferase reporter by the Dopamine receptor 2 (D2R) were not inhibited in the Ryk siRNA-containing cells, demonstrating that the Ryk siRNA-mediated inhibition was specific to the TCF pathway. This result provides indicates that Ryk plays an essential role in the activation of TCF pathway (See above).

Generation of Ryk Extra-cellular Domain Antibody.

The extracellular domain of Ryk was tagged with poly histadinene for protein purification. Mouse polyclonal antibody was generated for identification of the outside surface of Ryk. The polyclonal antibody recognized the over-expressed Ryk. This Ryk-specific antibody generation exemplifies the generation of an antibody suitable for targeting cancer cells.

REFERENCES CITED: Each of the following articles is incorporated herein by reference:

Behrens, J., von Kries, J. P., Kuhl, M., Bruhn, L., Wedlich, D., Grosschedl, R., and Birchmeier, W. (1996). Functional interaction of beta-catenin with the transcription factor LEF-1. Nature 382, 638-642.

Bhanot, P., Brink, M., Samos, C. H., Hsieh, J. C, Wang, Y., Macke, J. P., Andrew, D., Nathans, J., and Nusse, R. (1996). A new member of the frizzled family from Drosophila functions as a Wingless receptor. Nature 382, 225-230.

Bonkowsky, J. L., Yoshikawa, S., O'Keefe, D. D., Scully, A. L., and Thomas, J. B. (1999). Axon routing across the midline controlled by the Drosophila Derailed receptor. Nature 402, 540-544.

Brummelkamp, T. R., Bernards, R., and Agami, R. (2002). A system for stable expression of short interfering RNAs in mammalian cells. Science 296, 550-553.

Cadigan, K. M., and Nusse, R. (1997). Wnt signaling: a common theme in animal development. Genes Dev 11, 3286-3305.

Callahan, C. A., Muralidhar, M. G., Lundgren, S. E., Scully, A. L., and Thomas, J. B. (1995). Control of neuronal pathway selection by a Drosophila receptor protein-tyrosine kinase family member. Nature 376,171-174.

Castelo-Branco, G., Wagner, J., Rodriguez, F. J., Kele, J., Sousa, K., Rawal, N., Pasolli, H. A., Fuchs, E., Kitajewski, J., and Arenas, E. (2003). Differential regulation of midbrain dopaminergic neuron development by Wnt-1, Wnt-3a, and Wnt-5a. Proc Natl Acad Sci U S A 100,12747-12752.

Chenn, A., and Walsh, C. A. (2003). Increased neuronal production, enlarged forebrains and cytoarchitectural distortions in beta-catenin overexpressing transgenic mice. Cereb Cortex 13, 599-606.

Dura, J. M., Preat, T., and Tully, T. (1993). Identification of linotte, a new gene affecting learning and memory in Drosophila melanogaster. J Neurogenet 9,1-14.

Dura, J. M., Taillebourg, E., and Preat, T. (1995). The Drosophila learning and memory gene linotte encodes a putative receptor tyrosine kinase homologous to the human RYK gene product. FEBS Lett 370, 250-254.

Garcia-Castro, M. I., Marcelle, C, and Bronner-Fraser, M. (2002). Ectodermal Wnt Function As a Neural Crest Inducer. Science.

Halford, M. M., Armes, J., Buchert, M., Meskenaite, V., Grail, D., Hibbs, M. L., Wilks, A. F., Farlie, P. G., Newgreen, D. F., Hovens, C. M., and Stacker, S. A. (2000). Ryk-deficient mice exhibit craniofacial defects associated with perturbed Eph receptor crosstalk. Nat Genet 25,414-418.

Halford, M. M., and Stacker, S. A. (2001). Revelations of the RYK receptor. Bioessays 23, 34-45.

Hall, A. C, Lucas, F. R., and Salinas, P. C. (2000). Axonal remodeling and synaptic differentiation in the cerebellum is regulated by WNT-7a signaling. Cell 100, 525-535.

Hari, L., Brault, V., Kleber, M., Lee, H. Y., Ille, F., Leimeroth, R., Paratore, C, Suter, U., Kemler, R., and Sommer, L. (2002). Lineage-specific requirements of beta-catenin in neural crest development. J Cell Biol 159, 867-880.

Hovens, C. M., Stacker, S. A., Andres, A. C, Harpur, A. G., Ziemiecki, A., and Wilks, A. F. (1992). RYK, a receptor tyrosine kinase-related molecule with unusual kinase domain motifs. Proc Natl Acad Sci U S A 89,11818-11822.

Hsieh, J. C, Kodjabachian, L., Rebbert, M. L., Rattner, A., Smallwood, P. M., Samos, C. H., Nusse, R., Dawid, I. B., and Nathans, J. (1999). A new secreted protein that binds to Wnt proteins and inhibits their activities. Nature 398,431-436.

Huber, O., Kom, R., McLaughlin, J., Ohsugi, M., Herrmann, B. G., and Kemler, R. (1996). Nuclear localization of beta-catenin by interaction with transcription factor LEF-1.MechDev 59, 3-10.

Ikeya, M., Lee, S. M., Johnson, J. E., McMahon, A. P., and Takada, S. (1997). Wnt signalling required for expansion of neural crest and CNS progenitors. Nature 389, 966-970.

Ishitani, T., Ninomiya-Tsuji, J., and Matsumoto, K. (2003). Regulation of lymphoid enhancer factor I/T-cell factor by mitogen-activated protein kinase-related Nemo-like kinase-dependent phosphorylation in Wnt/beta-catenin signaling. Mol Cell Biol 23,1379-1389.

Ishitani, T., Ninomiya-Tsuji, J., Nagai, S., Nishita, M., Meneghini, M., Barker, N., Waterman, M., Bowerman, B., Clevers, H., Shibuya, H., and Matsumoto, K. (1999). The TAKI-NLK-MAPK-related pathway antagonizes signalling between beta-catenin and transcription factor TCF. Nature 399, 798-802.

Kamitori, K., Machide, M., Osumi, N., and Kohsaka, S. (1999). Expression of receptor tyrosine kinase RYK in developing rat central nervous system. Brain Res Dev Brain Res 114,149-160.

Krylova, O., Herreros, J., Cleverley, K. E., Ehler, E., Henriquez, J. P., Hughes, S. M., and Salinas, P. C. (2002). WNT-3, expressed by motoneurons, regulates terminal arborization of neurotrophin-3-responsive spinal sensory neurons. Neuron 35,1043-1056.

Lee, H. Y., Kleber, M., Hari, L., Brault, V., Suter, U., Taketo, M. M., Kemler, R., and Sommer, L. (2004). Instructive role of Wnt/beta-catenin in sensory fate specification in neural crest stem cells. Science 303,1020-1023.

Lewis, J. L., Bonner, J., Modrell, M., Ragland, J. W., Moon, R. T., Dorsky, R. I., and Raible, D. W. (2004). Reiterated Wnt signaling during zebrafish neural crest development. Development.

Lois, C, Hong, E. J., Pease, S., Brown, E. J., and Baltimore, D. (2002). Germline transmission and tissue-specific expression of transgenes delivered by lentiviral vectors. Science 295, 868-872.

Lucas, F. R., and Salinas, P. C. (1997). WNT-7a induces axonal remodeling and increases synapsin I levels in cerebellar neurons. Dev Biol 192, 31-44.

Lyuksyutova, A. I., Lu, C. C, Milanesio, N., King, L. A., Guo, N., Wang, Y., Nathans, J., Tessier-Lavigne, M., and Zou, Y. (2003). Anterior-posterior guidance of commissural axons by Wnt-frizzled signaling. Science 302,1984-1988.

McMahon, A. P., and Bradley, A. (1990). The Wnt-1 (int-) proto-oncogene is required for development of a large region of the mouse brain. Cell 62,1073-1085.

McMahon, A. P., Joyner, A. L., Bradley, A., and McMahon, J. A. (1992). The midbrain-hindbrain phenotype of Wnt-1 -/Wnt-1-mice results from step wise deletion of engrailed-expressing cells by 9.5 days postcoitum. Cell 69, 581-595.

Molenaar, M., van de Wetering, M., Oosterwegel, M., Peterson-Maduro, J., Godsave, S., Korinek, V., Roose, J., Destree, O., and Clevers, H. (1996). XTcf-3 transcription factor mediates beta-catenin-induced axis formation in Xenopus embryos. Cell 86, 391-399.

Moon, R. T., Bowerman, B., Boutros, M., and Perrimon, N. (2002). The promise and perils of Wnt signaling through beta-catenin. Science 296,1644-1646.

Moreau-Fauvarque, C, Taillebourg, E., Boissoneau, E., Mesnard, J., and Dura, J. M. (1998). The receptor tyrosine kinase gene linotte is required for neuronal pathway selection in the Drosophila mushroom bodies. Mech Dev 78,47-61.

Oates, A. C, Bonkovsky, J. L., Irvine, D. V., Kelly, L. E., Thomas, J. B., and Wilks, A. F. (1998). Embryonic expression and activity of doughnut, a second RYK homolog in Drosophila. Mech Dev 78,165-169.

Orford, K., Crockett, C, Jensen, J. P., Weissman, A. M., and Byers, S. W. (1997). Serine phosphorylation-regulated ubiquitination and degradation of beta-catenin. J Biol Chem 272, 24735-24738.

Orioli, D., Henkemeyer, M., Lemke, G., Klein, R., and Pawson, T. (1996). Sek4 and Nuk receptors cooperate in guidance of commissural axons and in palate formation. Embo J 75, 6035-6049.

Packard, M., Koo, E. S., Gorczyca, M., Sharpe, J., Cumberledge, S., and Budnik, V. (2002). The Drosophila Wnt, wingless, provides an essential signal for pre- and postsynaptic differentiation. Cell 111, 319-330.

Patthy, L. (2000). The WIF module. Trends Biochem Sci 25,12-13.

Peifer, M., Pai, L. M., and Casey, M. (1994). Phosphorylation of the Drosophila adherens junction protein Armadillo: roles for wingless signal and zeste-white 3 kinase. Dev Biol 166,543-556.

Peifer, M., and Polakis, P. (2000). Wnt signaling in oncogenesis and embryogenesis—a look outside the nucleus. Science 287,1606-1609.

Salic, A., Lee, E., Mayer, L., and Kirschner, M. W. (2000). Control of beta-catenin stability: reconstitution of the cytoplasmic steps of the wnt pathway in Xenopus egg extracts. Mol Cell 5, 523-532.

Simon, A. F., Boquet, I., Synguelakis, M., and Preat, T. (1998). The Drosophila putative kinase linotte (derailed) prevents central brain axons from converging on a newly described interhemispheric ring. Mech Dev 76,45-55.

Smit, L., Baas, A., Kuipers, J., Korswagen, H., Van De Wetering, M., and Clevers, H. (2004). Wnt activates the Takl/Nlk kinase pathway. J Biol Chem.

Sternberg, P. W., and Horvitz, H. R. (1988). lin-17 mutations of Caenorhabditis elegans disrupt certain asymmetric cell divisions. Dev Biol 130, 67-73.

Tamai, K., Semenov, M., Kato, Y., Spokony, R., Liu, C, Katsuyama, Y., Hess, F., Saint-Jeannet, J. P., and He, X. (2000). LDL-receptor-related proteins in Wnt signal transduction. Nature 407, 530-535.

Tanaka, M., Lu, W., Gupta, R., and Mayer, B. J. (1997). Expression of mutated Nek SH2/SH3 adaptor respecifies mesodermal cell fate in Xenopus laevis development. Proc Natl Acad Sci U S A 94,4493-4498.

Thomas, K. R., and Capecchi, M. R. (1990). Targeted disruption of the murine int-1 proto-oncogene resulting in severe abnormalities in midbrain and cerebellar development. Nature 346, 847-850.

Van Es, J. H., Barker, N., and Clevers, H. (2003). You Wnt some, you lose some: oncogenes in the Wnt signaling pathway. Curr Opin Genet Dev 13, 28-33.

Veeman, M. T., Axelrod, J. D., and Moon, R. T. (2003). A second canon. Functions and mechanisms of beta-catenin-independent Wnt signaling. Dev Cell 5, 367-377.

Wehrli, M., Dougan, S. T., Caldwell, K., O'Keefe, L., Schwartz, S., Vaizel-Ohayon, D., Schejter, E., Tomlinson, A., and DiNardo, S. (2000). arrow encodes an LDL-receptor-related protein essential for Wingless signalling. Nature 407, 527-530.

Willert, K., and Nusse, R. (1998). Beta-catenin: a key mediator of Wnt signaling. Curr Opin Genet Dev 8, 95-102.

Wodarz, A., and Nusse, R. (1998). Mechanisms of Wnt signaling in development. Annu Rev Cell Dev Biol 14, 59-88.

Wong, H. C, Bourdelas, A., Krauss, A., Lee, H. J., Shao, Y., Wu, D., Mlodzik, M., Shi, D. L., and Zheng, J. (2003). Direct binding of the PDZ domain of Dishevelled to a conserved internal sequence in the C-terminal region of Frizzled. Mol Cell 12,1251-1260.

Yee, K., Bishop, T. R., Mather, C, and Zon, L. I. (1993). Isolation of a novel receptor tyrosine kinase cDNA expressed by developing erythroid progenitors. Blood 82,1335-1343.

Yoshikawa, S., McKinnon, R. D., Kokel, M., and Thomas, J. B. (2003). Wnt-mediated axon guidance via the Drosophila Derailed receptor. Nature 422, 583-588.

Yost, C, Torres, M., Miller, J. R., Huang, E., Kimelman, D., and Moon, R. T. (1996). The axis-inducing activity, stability, and subcellular distribution of beta-catenin is regulated in Xenopus embryos by glycogen synthase kinase 3. Genes Dev 10,1443-1454.

Although the invention has been described with reference to the above example, it will be understood that modifications and variations are encompassed within the spirit and scope of the invention. Accordingly, the invention is limited only by the following claims.

1. A method of inducing neurite outgrowth, comprising contacting a cell with a Wnt polypeptide, wherein the cell comprises a neuronal cell or a neuronal precursor cell.

2. The method of claim 1, wherein the Wnt polypeptide comprises a Wnt3a, Wnt1, or Wnt4 polypeptide.

3. The method of claim 2, wherein the Wnt3a polypeptide comprises a polypeptide having an amino acid sequence set forth in SEQ ID NO:2, SEQ ID NO:4, or a functional fragment of said polypeptide that selectively binds Ryk and Frizzled; wherein the Wnt1 polypeptide comprises a polypeptide having an amino acid sequence set forth in SEQ ID NO:6, SEQ ID NO:8, or a functional fragment of said polypeptide that selectively binds Ryk and Frizzled; or wherein the Wnt4 polypeptide comprises a polypeptide having an amino acid sequence set forth in SEQ ID NO:10, SEQ ID NO:12, or a functional fragment of said polypeptide that selectively binds Ryk and Frizzled.

4-5. (canceled)

6. The method of claim 1, further comprising contacting the cell with a soluble Ryk polypeptide.

7. The method of claim 6, wherein the soluble Ryk comprises an extracellular domain of a Ryk polypeptide.

8. The method of claim 7, wherein the extracellular domain of Ryk comprises about amino acid residues 42-224 as set forth in SEQ ID NO:14 or about amino acid residues 36-211 as set forth in SEQ ID NO:16.

9. The method of claim 1, wherein the cell is a mammalian cell.

10. The method of claim 1, wherein the cell is a human cell.

11. The method of claim 10, wherein the cell is from a subject having a neuronal disorder.

12. The method of claim 11, wherein the neuronal disorder comprises a neurodegenerative disease or traumatic nerve injury.

13-14. (canceled)

15. The method of claim 1, which comprises contacting the cell in vivo, or ex vivo.

16. (canceled)

17. A method of ameliorating a neuronal disorder in a subject, comprising administering to the subject a therapeutically effective amount of a Wnt polypeptide, a soluble Ryk polypeptide, or a combination thereof.

18. The method of claim 17, wherein the Wnt polypeptide comprises a Wnt3a, Wnt1, or Wnt4 polypeptide.

19. The method of claim 17, further comprising administering a soluble Ryk polypeptide to the subject.

20. The method of claim 19, wherein the soluble Ryk polypeptide comprises an extracellular domain of Ryk.

21-22. (canceled)

23. The method of claim 17, wherein the subject is a human.

24-26. (canceled)

27. A method of inducing growth of hematopoietic stem cells, comprising contacting a hematopoietic stem cell with a Wnt polypeptide and a soluble Ryk polypeptide.

28. The method of claim 27, wherein the Wnt polypeptide is a Wnt3a polypeptide.

29. The method of claim 27, wherein the soluble Ryk polypeptide comprises an extracellular domain of Ryk.

30. (canceled)

31. The method of claim 27, wherein the cell is a human cell.

32. The method of claim 27, wherein the contacting comprises contacting in vivo, or ex vivo.

33. (canceled)

34. A composition comprising a soluble Ryk polypeptide.

35. The composition of claim 34, wherein the soluble Ryk comprises an extracellular domain of Ryk.

36. The composition of claim 35, wherein the extracellular domain of Ryk comprises about amino acid residues 42-224 as set forth in SEQ ID NO:14 or about amino acid residues 36-211 as set forth in SEQ ID NO:16.

37. The composition of claim 34, further comprising a Wnt polypeptide.

38. The composition of claim 37, wherein the Wnt polypeptide comprises a Wnt3a, Wnt1, or Wnt4 polypeptide.

39-45. (canceled)

46. A kit, comprising the soluble Ryk polypeptide and the Wnt polypeptide of claim 37.

47-48. (canceled)

49. A method of modulating an effect of Wnt on a cell, comprising contacting the cell with soluble Ryk polypeptide that selectively binds Wnt or Frizzled, whereby selective binding of the soluble Ryk polypeptide affects Ryk mediated Wnt signal transduction in the cell, thereby modulating an effect of Wnt on a cell.

50. The method of claim 49, wherein the soluble Ryk polypeptide can specifically interact with Wnt and Frizzled, and not Disheveled, thereby affecting Ryk mediated signal transduction.

51. (canceled)

52. The method of claim 49, wherein the cell is a neuronal cell or neuronal precursor cell, or a cancer cell.

53. (canceled)

54. The method of claim 49, wherein the agent is a Wnt agonist, or a Wnt antagonist.

55. (canceled)

56. A method of identifying an agent that modulates Ryk mediated signal transduction, comprising:
contacting a sample comprising Ryk and Frizzled with a test agent, under conditions suitable for binding of Wnt to Ryk and Frizzled;
detecting a change in Ryk mediated signal transduction due to selective binding of the agent to Ryk and Frizzled, thereby identifying an agent that modulates Ryk mediated signal transduction.

57. The method of claim 56, wherein the sample comprises a cell expressing Ryk and Frizzled.

58. The method of claim 57, wherein Ryk and Frizzled are endogenous to the cell.

59. The method of claim 57, wherein the cell is a neuronal or neuronal precursor cell or a cancer cell.

60. (canceled)

61. The method of claim 56, wherein the detecting a change in Ryk mediated signal transduction comprises detecting a change in TCF (T-Cell specific factor) activation in the presence of the test agent as compared to TCF activation in the absence of the test agent.

62. The method of claim 56, wherein the test agent comprises a combinatorial library of test agents.

63. (canceled)

64. The method of claim 56, wherein the sample further comprises a Wnt polypeptide.

65. The method of claim 64, wherein the agent is a Wnt agonist, which increases Ryk mediated signal transduction, or a Wnt antagonist, which reduces or inhibits Ryk mediated signal transduction.

66. (canceled)

67. An agent identified by the method of claim 56.

68. A method of identifying an agent that modulates a specific interaction of Ryk and Frizzled, comprising:
contacting a sample comprising Ryk and Frizzled with a test agent, under conditions suitable for formation of a Ryk/Frizzled complex suitable for Ryk mediated signal transduction; and
detecting a change in the complex in the presence of the test agent as compared to complex formation in the absence of the test agent, thereby identifying an agent that modulates a specific interaction between Ryk and Frizzled.

69-71. (canceled)

72. The method of claim 68, further comprising detecting a change in TCF activation in the presence of the test agent as compared to TCF activation in the absence of the test agent.

73-74. (canceled)

75. A method of identifying an agent that modulates a specific interaction of Ryk and Disheveled, comprising:
contacting a sample comprising Ryk and Disheveled with a test agent, under conditions suitable for formation of a Ryk/Disheveled complex suitable for Ryk mediated signal transduction; and
detecting a change in formation of the complex in the presence of the test agent as compared to complex formation in the absence of the test agent, thereby identifying an agent that modulates a specific interaction between Ryk and Disheveled.

76-78. (canceled)

79. The method of claim 75, wherein the sample further comprises Frizzled, a Wnt polypeptide, or both.

80-83. (canceled)


SEQUENCE LISTING (30 sequences)

SEQ ID NO: 1 - DNA, 2932 bp, Homo sapiens
agctcccagggcccggccccccccggcgctcacgctctcggggcggactcccggccctccgcgccctcccgcgcggcgatggccccactcggatacttcttactcctctgcagcctgaagcaggctctgggcagctacccgatctggtggtcgctggctgttgggccacagtattcctccctgggctcgcagcccatcctgtgtgccagcatcccgggcctggtccccaagcagctccgcttctgcaggaactacgtggagatcatgcccagcgtggccgagggcatcaagattggcatccaggagtgccagcaccagttccgcggccgccggtggaactgcaccaccgtccacgacagcctggccatcttcgggcccgtgctggacaaagctaccagggagtcggcctttgtccacgccattgcctcagccggtgtggcctttgcagtgacacgctcatgtgcagaaggcacggccgccatctgtggctgcagcagccgccaccagggctcaccaggcaagggctggaagtggggtggctgtagcgaggacatcgagtttggtgggatggtgtctcgggagttcgccgacgcccgggagaaccggccagatgcccgctcagccatgaaccgccacaacaacgaggctgggcgccaggccatcgccagccacatgcacctcaagtgcaagtgccacgggctgtcgggcagctgcgaggtgaagacatgctggtggtcgcaacccgacttccgcgccatcggtgacttcctcaaggacaagtacgacagcgcctcggagatggtggtggagaagcaccgggagtcccgcggctgggtggagaccctgcggccgcgctacacctacttcaaggtgcccacggagcgcgacctggtctactacgaggcctcgcccaacttctgcgagcccaaccctgagacgggctccttcggcacgcgcgaccgcacctgcaacgtcagctcgcacggcatcgacggctgcgacctgctgtgctgcggccgcggccacaacgcgcgagcggagcggcgccgggagaagtgccgctgcgtgttccactggtgctgctacgtcagctgccaggagtgcacgcgcgtctacgacgtgcacacctgcaagtaggcaccggccgcggctccccctggacggggcgggccctgcctgagggtgggcttttccctgggtggagcaggactcccacctaaacggggcagtactcctccctgggggcgggactcctccctgggggtggggctcctacctgggggcagaactcctacctgaaggcagggctcctccctggagccagtgtctcctctctggtggctgggctgctcctgaatgaggcggagctccaggatggggaggggctctgcgttggcttctccctggggacggggctcccctggacagaggcggggctacagattgggcggggcttctcttgggtgggacagggcttctcctgcgggggcgaggcccctcccagtaagggcgtggctctgggtgggcggggcactaggtaggcttctacctgcaggcggggctcctcctgaaggaggcggggctctaggatggggcacggctctggggtaggctgctccctgagggcggagcgcctccttaggagtggggttttatggtggatgaggcttcttcctggatggggcagagcttctcctgaccagggcaaggccccttccacgggggctgtggctctgggtgggcgtggcctgcataggctccttcctgtgggtggggcttctctgggaccaggctccaatggggcggggcttctctccgcgggtgggactcttccctgggaaccgccctcctgattaaggcgtggcttctgcaggaatcccggctccagagcaggaaattcagcccaccagccacctcatccccaaccccctgtaaggttccatccacccctgcgtcgagctgggaaggttccatgaagcgagtcgggtccccaacccgtgcccctgggatccgagggcccctctccaagcgcctggctttggaatgctccaggcgcgccgacgcctgtgccaccccttcctcagcctggggtttgaccacccacctgaccaggggccctacctggggaaagcctgaagggcctcccagcccccaaccccaagaccaagcttagtcctgggagaggacagggacttcgcagaggcaagcgaccgaggccctcccaaagaggcccgccctgcccgggctcccacaccgtcaggtactcctgccagggaactggcctgctgcgccccaggccccgcccgtctctgctctgctcagctgcgcccccttctttgcagctgcccagcccctcctccctgccctcgggtctccccacctgcactccatccagctacaggagagatagaagcctctcgtcccgtccctccctttcctccgcctgtccacagccccttaagggaaaggtaggaagagaggtccagccccccaggctgcccagagctgctggtctcatttgggggcgttcgggaggtttggggggcatcaaccccccgactgtgctgctcgcgaaggtcccacagccctgagatgggccggcccccttcctggcccctcatggcgggactggagaaatggtccgctttcctggagccaatggcccggcccctcctgactcatccgcctggcccgggaatgaatggggaggccgctgaacccacccggcccatatccctggttgcctcatggccagcgcccctcagcctctgccactgtgaaccggctcccaccctcaaggtgcggggagaagaagcggccaggcggggcgccccaagagcccaaaagagggcacaccgccatcctctgcctcaaattctgcgtttttggttttaatgttatatctgatgctgctatatccactgtccaacgg

SEQ ID NO: 2 - protein, 352 aa, Homo sapiens
MAPLGYFLLLCSLKQALGSYPIWWSLAVGPQYSSLGSQPILCASIPGLVPKQLRFCRNYVEIMPSVAEGIKIGIQECQHQFRGRRWNCTTVHDSLAIFGPVLDKATRESAFVHAIASAGVAFAVTRSCAEGTAAICGCSSRHQGSPGKGWKWGGCSEDIEFGGMVSREFADARENRPDARSAMNRHNNEAGRQAIASHMHLKCKCHGLSGSCEVKTCWWSQPDFRAIGDFLKDKYDSASEMVVEKHRESRGWVETLRPRYTYFKVPTERDLVYYEASPNFCEPNPETGSFGTRDRTCNVSSHGIDGCDLLCCGRGHNARAERRREKCRCVFHWCCYVSCQECTRVYDVHTCK

SEQ ID NO: 3 - DNA, 2814 bp, Mus musculus
gaattcatgtcttacggtcaaggcagagggcccagcgccactgcagccgcgccacctcccagggccgggccagcccaggcgtccgcgctctcggggtggactccccccgctgcgcgctcaagccggcgatggctcctctcggatacctcttagtgctctgcagcctgaagcaggctctgggcagctacccgatctggtggtccttggctgtgggaccccagtactcctctctgagcactcagcccattctctgtgccagcatcccaggcctggtaccgaagcagctgcgcttctgcaggaactacgtggagatcatgcccagcgtggctgagggtgtcaaagcgggcatccaggagtgccagcaccagttccgaggccggcgttggaactgcaccaccgtcagcaacagcctggccatctttggccctgttctggacaaagccacccgggagtcagcctttgtccatgccatcgcctccgctggagtagctttcgcagtgacacgctcctgtgcagagggatcagctgctatctgtgggtgcagcagccgcctccagggctccccaggcgagggctggaagtggggcggctgtagtgaggacattgaatttggaggaatggtctctcgggagtttgccgatgccagggagaaccggccggatgcccgctctgccatgaaccgtcacaacaatgaggctgggcgccaggccatcgccagtcacatgcacctcaagtgcaaatgccacgggctatctggcagctgtgaagtgaagacctgctggtggtcgcagccggacttccgcaccatcggggatttcctcaaggacaagtatgacagtgcctcggagatggtggtagagaaacaccgagagtctcgtggctgggtggagaccctgaggccacgttacacgtacttcaaggtgccgacagaacgcgacctggtctactacgaggcctcacccaacttctgcgaacctaaccccgaaaccggctccttcgggacgcgtgaccgcacctgcaatgtgagctcgcatggcatagatgggtgcgacctgttgtgctgcgggcgcgggcataacgcgcgcactgagcgacggagggagaaatgccactgtgttttccattggtgctgctacgtcagctgccaggagtgcacacgtgtctatgacgtgcacacctgcaagtaggagagctcctaacacgggagcagggttcattccgaggggcaaggttcctacctgggggcggggttcctacttggaggggtctcttacttggggactcggttcttacttgagggcggagatcctacctgtgagggtctcatacctaaggacccggtttctgccttcagcctgggctcctatttgggatctgggttcctttttaggggagaagctcctgtctgggatacgggtttctgcccgagggtggggctccacttggggatggaattccaatttgggccggaagtcctacctcaatggcttggactcctctcttgacccgacagggctcaaatggagacaggtaagctactccctcaactaggtggggttcgtgcggatgggtgggaggggagagattagggtccctcctcccagaggcactgctctatctagatacatgagagggtgcttcagggtgggccctatttgggcttgaggatcccgtgggggcggggcttcaccccgactgggtggaacttttggagacccccttccactggggcaaggcttcactgaagactcatgggatggagctccacggaaggaggagttcctgagcgagcctgggctctgagcaggccatccagctcccatctggcccctttccagtcctggtgtaaggttcaacctgcaagcctcatctgcgcagagcaggatctcctggcagaatgaggcatggagaagaactcaggggtgataccaagacctaacaaaccccgtgcctgggtacctcttttaaagctctgcaccccttcttcaagggctttcctagtctccttggcagagctttcctgaggaagatttgcagtcccccagagttcaagtgaacacccatagaacagaacagactctatcctgagtagagagggttctctaggaatctctatggggactgctaggaaggatcctgggcatgacagcctcgtatgatagcctgcatccgctctgacacttaatactcagatctcccgggaaacccagctcatccggtccgtgatgtccatgccccaaatgcctcagagatgttgcctcactttgagttgtatgaacttcggagacatggggacacagtcaagccgcagagccagggttgtttcaggacccatctgattccccagagcctgctgttgaggcaatggtcaccagatccgttggccaccaccctgtcccgagcttctctagtgtctgtctggcctggaagtgaggtgctacatacagcccatctgccacaagagcttcctgattggtaccactgtgaaccgtccctccccctccagacaggggaggggatgtggccatacaggagtgtgcccggagagcgcggaaagaggaagagaggctgcacacgcgtggtgactgactgtcttctgcctggaactttgcgttcgcgcttgtaactttattttcaatgctgctatatccacccaccactggatttagacaaaagtgattttctttttttttttttcttttctttctatgaaagaaattattttagtttatagtatgtttgtttcaaataatggggaaagtaaaaagagagaaaaaaaaaaaaaaaaaaaaaaaaaaaaaaaaaaaaaaaaaa

SEQ ID NO: 4 - protein, 352 aa, Mus musculus
MAPLGYLLVLCSLKQALGSYPIWWSLAVGPQYSSLSTQPILCASIPGLVPKQLRFCRNYVEIMPSVAEGVKAGIQECQHQFRGRRWNCTTVSNSLAIFGPVLDKATRESAFVHAIASAGVAFAVTRSCAEGSAAICGCSSRLQGSPGEGWKWGGCSEDIEFGGMVSREFADARENRPDARSAMNRHNNEAGRQAIASHMHLKCKCHGLSGSCEVKTCWWSQPDFRTIGDFLKDKYDSASEMVVEKHRESRGWVETLRPRYTYFKVPTERDLVYYEASPNFCEPNPETGSFGTRDRTCNVSSHGIDGCDLLCCGRGHNARTERRREKCHCVFHWCCYVSCQECTRVYDVHTCK

SEQ ID NO: 5 - DNA, 4522 bp, Homo sapiens
cagctgagtgaggcgggcgcgcgtgggagggtgtcccaaggggaggggtccgcggccagtgcaggcccggaggcgggggccaccgggcagggggcgggggtgagccccgacggccaacccgtcagctctcggctcagacgggcgggaaccacagccccgctcgctgcccattgtctgcgcccctaaccggtgcgccctggtgccacagtgcggcccggaggggcagcctcctcccgtcacttcagccagcgccgcaactataagaggcggtgccgcccgccgtggccgcctcagcccaccagccgggaccgcgagccatgctgtccgccgcccgcccccagggttgttaaagccagactgcgaactctcgccactgccgccaccgccgcgtcccgtcccaccgtcgcgggcaacaaccaaagtcgccgcaactgcagcacagagcgggcaaagccaggcaggccatggggctctgggcgctgttgcctggctgggtttctgctacgctgctgctggcgctggccgctctgcccgcagccctggctgccaacagcagtggccgatggtggtaagtgagctggtgcggggtcgccacttgtcccgcggcacagagccaggggccaaccctacccagctcccacgctctgggatccgtctgccgacaggctccctccccgctctgacttccctccgcgacaccgaagggcgatctggcatgaaactgccccagactccagctctgtacaagtggggcgaatgatccgcccgcggaggcctaagataccccaggcagggagcccactctcatctagcaccgcccttcccctttgagcgccaactccagcctcacggcggtggctcaccacaggtttccccacctcgggaagtgaagggccaggagttcgcctagaaaggaggggagaagagggtgggactcctaagcatttcacgccttgggtgggcaagaactgcaggccatgattatctcgctcaggctgaccggaagaggctcggagatccaaggtagacactcggtctccgggtacctcctctgtccagtctccggacctagggctcaggcgagcagccctgggactactgggcacacacaagtctggacgcccagttctttcaaattagtgagcctgggagagcgggtattattaatctcccgccattctctccagccacatacccccaggaagaggaccgggtggcacagtttttatggttagggtgcggatccccttcctgagcctgagctatcatacgtcccaccaggggtattgtgaacgtagcctcctccacgaacctgcttacagactccaagagtctgcaactggtactcgagcccagtctgcagctgttgagccgcaaacagcggcgcctgatacgccaaaatccggggatcctgcacagcgtgagtggggggctgcagagtgccgtgcgcgagtgcaagtggcagttccggaatcgccgctggaactgtcccactgctccagggccccacctcttcggcaagatcgtcaaccgaggtgggtgcccaggaaggcgacgcttccgggagcaggggaaacgcggggtcacccccagggcatgggcgggcgagttcagagaaggtgtcccaggcgcctggagggtcacacaatcaaccttgccaagtgcctcgtgcccagcgccagctcggggccagacttctaccaggcgttttccagccgtgcaccctggaaacgaagcttaacttttctgagctactgccccagataaagaaagtttcgggtcgcggacgccggctgaccgccgctttcccccagcctctctcaaaagcgcctgggaagctgctctctgcaggcgtgtgtctggcctctcgcccagcaaggcttgcaccgccaaaatgggccgaaagttttgggctgcgaagaagtcttggggatgtatggttcttccgctcccctctcttcggtttgtctctctggggctgctccacttccgctatcgagccaaaatgcgccctagaatctcccagtaaggtgtgattacgcccgtggacgtggctgcgtgcccacgcacctgctttctctactagccctagagaccagctttccagcactgccggccctggtcctcaggactcaaagtgcggagtcgggggtgggattccggtcccaagcccttcatgagggtgctggccgcgccccgcgtaccccctcgctgatccccgctcccttctcccacaggctgtcgagaaacggcgtttatcttcgctatcacctccgccggggtcacccattcggtggcgcgctcctgctcagaaggttccatcgaatcctgcacgtgtgactaccggcggcgcggccccgggggccccgactggcactgggggggctgcagcgacaacattgacttcggccgcctcttcggccgggagttcgtggactccggggagaaggggcgggacctgcgcttcctcatgaaccttcacaacaacgaggcaggccgtacggtgagctttgagaggctccgcaccctaagcggagcggcaggggccaacctcgggctggggaagtgacggtcggtgagataaggcaaggggcaccaggagagggcgtcctgggagagccggaggcttggaacgaagacggagaatagaggagacagtggctgagggcaaaggtatgtctggcccgcggacaggtagaagaggttgcaaatcaagcacagtctcttcgctgtacagattcgaaaaataagcctgagaggccgagactgactcgccgcggcggagcagggttgggcagggtttccaaatctcagcggaacatttcgcgcctcccttcccctgggctcagctaggcctgggcctttgctgaggtccggcccccgtggcgtccgggagagggcagtgtctgggagggtgactctggcccggtgccctgggacactctttcttcccctatccccgcagaccgtattctccgagatgcgccaggagtgcaagtgccacgggatgtccggctcatgcacggtgcgcacgtgctggatgcggctgcccacgctgcgcgccgtgggcgatgtgctgcgcgaccgcttcgacggcgcctcgcgcgtcctgtacggcaaccgcggcagcaaccgcgcttcgcgagcggagctgctgcgcctggagccggaagacccggcccacaaaccgccctccccccacgacctcgtctacttcgagaaatcgcccaacttctgcacgtacagcggacgcctgggcacagcaggcacggcagggcgcgcctgtaacagctcgtcgcccgcgctggacggctgcgagctgctctgctgcggcaggggccaccgcacgcgcacgcagcgcgtcaccgagcgctgcaactgcaccttccactggtgctgccacgtcagctgccgcaactgcacgcacacgcgcgtactgcacgagtgtctgtgaggcgctgcgcggactcgcccccaggaacgctctcctcgagccctcccccaaacagactcgctagcactcaagacccggttattcgcccacccgagtacctccagtcacactccccgcggttcatacgcatcccatctctcccacttcctcctacctggggactcctcaaaccacttgcctggggcggcatgaaccctcttgccatcctgatggacctgccccggacctaacctccctccctctccgcgggagaccccttgttgcactgccccctgcttggccaggaggtgagagaaggatgggtcccctccgccatggggtcggctcctgatggtgtcattctgcctgctccatcgcgccagcgacctctctgcctctcttcttcccctttgtcctgcgttttctccgggtcctcctaagtcccttcctattctcctgccatgggtgcagaccctgaacccacacctgggcatcagggcctttctcctccccacctgtagctgaagcaggaggttacagggcaaaagggcagctgtgatgatgtgggaatgaggttgggggaaccagcagaaatgcccccattctcccagtctctgtcgtggagccattgaacagctgtgagccatgcctccctgggccacctcctaccccttcctgtcctgcctcctcatcagtgtgtaaataatttgcactgaaacgtggatacagagccacgagtttggatgttgtaaataaaactatttattgtgctgggtcccagcctggtttgcaaagaccacctccaacccaacccaatccctctccactcttctctcctttctccctgcagccttttctggtccctcttctctcctcagtttctcaaagatgcgtttgcctcctggaatcagtatttccttccactgtagctattagcggctcctcgcccccaccagtgtagcatcttcctctgcagaataaaatctctatttttatcgatgacttggtggcttttccttgaatccagaacacaaccttgtttgtggtgtcccctatcctccccttttaccactcccagcttggaagctt

SEQ ID NO: 6 - protein, 370 aa, Homo sapiens
MGLWALLPGWVSATLLLALAALPAALAANSSGRWWGIVNVASSTNLLTDSKSLQLVLEPSLQLLSRKQRRLIRQNPGILHSVSGGLQSAVRECKWQFRNRRWNCPTAPGPHLFGKIVNRGCRETAFIFAITSAGVTHSVARSCSEGSIESCTCDYRRRGPGGPDWHWGGCSDNIDFGRLFGREFVDSGEKGRDLRFLMNLHNNEAGRTTVFSEMRQECKCHGMSGSCTVRTCWMRLPTLRAVGDVLRDRFDGASRVLYGNRGSNRASRAELLRLEPEDPAHKPPSPHDLVYFEKSPNFCTYSGRLGTAGTAGRACNSSSPALDGCELLCCGRGHRTRTQRVTERCNCTFHWCCHVSCRNCTHTRVLHECL

SEQ ID NO: 7 - DNA, 2463 bp, Mus musculus
gccacagcttcgctcgccactcattgtctgtggccctgaccagtgcgccctggtgcttttagtgccgcccgggcccggaggggcagcctcttctcactgcagtcagcgccgcaactataagaggcggtgcctcccgcagtggctgcttcagcccagcagccaggacagcgaaccatgctgcctgcggcccgcctccagacttattagagccagcctgggaactcgcatcactgccctcaccgctgtgtccagtcccaccgtcgcggacagcaaccacagtcgtcagaaccccagcacagaaccagcaaggccaggcaggccatggggctctgggcgctgctgcccagctgggtttctactacgttgctactggcactgaccgctctgcccgcagccctagctgccaacagtagtggccgatggtggggcatcgtgaacatagcctcctccacgaacctgttgacggattccaagagcctgcagctggtgctcgagcccagtctgcagctgctgagccgcaagcagcggcgactgatccgacagaacccggggatcctgcacagcgtgagtggagggctccagagcgccgtgcgagagtgcaaatggcaattccgaaaccgccgctggaactgccccactgctccggggccccacctcttcggcaagatcgtcaaccgaggctgccgagaaacagcgttcatcttcgcaatcacctccgccggggtcacacattccgtggcgcgctcctgctccgaaggctccatcgagtcctgcacctgcgactaccggcggcgcggccctgggggccccgactggcactgggggggctgcagtgacaacatcgattttggtcgcctctttggccgagagttcgtggactccggggagaaggggcgggacctacgcttcctcatgaaccttcacaacaacgaggcagggcgaacgaccgtgttctctgagatgcgccaagagtgcaaatgccacgggatgtccggctcctgcacggtgcgcacgtgttggatgcggctgcccacgctgcgcgctgtgggcgacgtgctgcgcgaccgcttcgacggtgcctcccgcgtcctttacggcaacagaggcagcaaccgcgcctcgcgggcggagctgctgcgcctggagcccgaagaccccgcgcacaagcctccctcccctcacgacctcgtctacttcgagaaatcgcccaacttctgcacgtacagtggccgcctgggcacagctggcacagctggacgagcttgcaacagctcgtctcccgcgctggacggctgtgagctgctgtgctgtggccgaggccaccgcacgcgcacgcagcgcgtcacggagcgctgcaactgcaccttccactggtgctgccacgtcagctgccgcaactgcacgcacacgcgcgttctgcacgagtgtctatgaggtgccgcgcctccgggaacgggaacgctctcttccagttctcagacacactcactggtcctgatgtttgcccaccctaccgcgtccagccacagtcccagggttcatagcgatccatctctcccacctcctacctggggactcctgaaaccacttgcctgagtcggctcgaacccttttgccatcctgagggccctgacccagcctacctccctccctctttgagggagactccttttgcactgccccccaatttggccagagggtgagagaaagattcttcttctggggtgggggtggggaggtcaactcttgaaggtgttgcggttcctgatgtattttgcgctgtgacctctttgggtattatcacctttccttgtctctcaggtccctataggtcccttgagttctctaaccagcacctctgggcttcaaggcctttcccctcccacctgtagctgaagagtttccgagttgaaagggcacggaaagctaagtgggaaaggaggttgctggacccagcagcaaaaccctacattctccttgtctctgcctcggagccattgaacagctgtgaaccatgcctccctcagcctcctcccaccccttcctgtcctgcctcctcatcactgtgtaaataatttgcaccgaaatgtggccgcagagccacgcgttcggttatgtaaataaaactatttattgtgctgggttccagcctgggttgcagagaccaccctcaccccacctcactgctcctctgttctgcttgccagtccttttgttatccgaccttttttctcttttacccagcttctcataggcgcccttgcccaccggatcagtatttccttccactgtagctattagtggctcctcgcccccaccaatgtagtatcttcctctgaggaataaaatatctatttttaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 8 - protein, 370 aa, Mus musculus
MGLWALLPSWVSTTLLLALTALPAALAANSSGRWWGIVNIASSTNLLTDSKSLQLVLEPSLQLLSRKQRRLIRQNPGILHSVSGGLQSAVRECKWQFRNRRWNCPTAPGPHLFGKIVNRGCRETAFIFAITSAGVTHSVARSCSEGSIESCTCDYRRRGPGGPDWHWGGCSDNIDFGRLFGREFVDSGEKGRDLRFLMNLHNNEAGRTTVFSEMRQECKCHGMSGSCTVRTCWMRLPTLRAVGDVLRDRFDGASRVLYGNRGSNRASRAELLRLEPEDPAHKPPSPHDLVYFEKSPNFCTYSGRLGTAGTAGRACNSSSPALDGCELLCCGRGHRTRTQRVTERCNCTFHWCCHVSCRNCTHTRVLHECL

SEQ ID NO: 9 - DNA, 2049 bp, Homo sapiens
agccgctgccccgggccgggcgcccgcggcggcaccatgagtccccgctcgtgcctgcgttcgctgcgcctcctcgtcttcgccgtcttctcagccgccgcgagcaactggctgtacctggccaagctgtcgtcggtggggagcatctcagaggaggagacgtgcgagaaactcaagggcctgatccagaggcaggtgcagatgtgcaagcggaacctggaagtcatggactcggtgcgccgcggtgcccagctggccattgaggagtgccagtaccagttccggaaccggcgctggaactgctccacactcgactccttgcccgtcttcggcaaggtggtgacgcaagggactcgggaggcggccttcgtgtacgccatctcttcggcaggtgtggcctttgcagtgacgcgggcgtgcagcagtggggagctggagaagtgcggctgtgacaggacagtgcatggggtcagcccacagggcttccagtggtcaggatgctctgacaacatcgcctacggtgtggccttctcacagtcgtttgtggatgtgcgggagagaagcaagggggcctcgtccagcagagccctcatgaacctccacaacaatgaggccggcaggaaggccatcctgacacacatgcgggtggaatgcaagtgccacggggtgtcaggctcctgtgaggtaaagacgtgctggcgagccgtgccgcccttccgccaggtgggtcacgcactgaaggagaagtttgatggtgccactgaggtggagccacgccgcgtgggctcctccagggcactggtaccacgcaacgcacagttcaagccgcacacagatgaggacctggtgtacttggagcctagccccgacttctgtgagcaggacatgcgcagcggcgtgctgggcacgaggggccgcacatgcaacaagacgtccaaggccatcgacggctgtgagctgctgtgctgtggccgcggcttccacacggcgcaggtggagctggctgaacgctgcagctgcaaattccactggtgctgcttcgtcaagtgccggcagtgccagcggctcgtggagttgcacacgtgccgatgaccgcctgcctagccctgcgccggcaaccacctagtggcccagggaaggccgataatttaaacagtctcccaccacctaccccaagagatactggttgtattttttgttctggtttggtttttgggtcctcatgttatttattgccgaaaccaggcaggcaaccccaagggcaccaaccagggcctccccaaagcctgggcctttgtggctgccactgaccaaagggaccttgctcgtgccgctggctgcccgcatgtggctgccactgaccactcagttgttatctgtgtccgtttttctacttgcagacctaaggtggagtaacaaggagtattaccaccacatggctactgaccgtgtcatcggggaagagggggccttatggcagggaaaataggtaccgacttgatggaagtcacaccctctggaaaaaagaactcttaactctccagcacacatacacatggactcctggcagcttgagcctagaagccatgtctctcaaatgccctgagaaagggaacaagcagataccaggtcaagggcaccaggttcatttcagcccttacatggacagctagaggttcgatatctgtgggtccttccaggcaagaagagggagatgagagcaagagacgactgaagtcccaccctagaacccagcctgccccagcctgcccctgggaagaggaaacttaaccactccccagacccacctaggcaggcatataggctgccatcctggaccagggatcccggctgtgcctttgcagtcatgcccgagtcacctttcacagcgctgttcctccatgaaactgaaaaacacacacacacacacacacacacacacacacacacacacacacacggacacacacacacacctgcgagagagagggaggaaagggctgtgcctttgcagtcatgcccgagtcacctttcacagcactgttcctc

SEQ ID NO: 10 - protein, 351 aa, Homo sapiens
MSPRSCLRSLRLLVFAVFSAAASNWLYLAKLSSVGSISEEETCEKLKGLIQRQVQMCKRNLEVMDSVRRGAQLAIEECQYQFRNRRWNCSTLDSLPVFGKVVTQGTREAAFVYAISSAGVAFAVTRACSSGELEKCGCDRTVHGVSPQGFQWSGCSDNIAYGVAFSQSFVDVRERSKGASSSRALMNLHNNEAGRKAILTHMRVECKCHGVSGSCEVKTCWRAVPPFRQVGHALKEKFDGATEVEPRRVGSSRALVPRNAQFKPHTDEDLVYLEPSPDFCEQDMRSGVLGTRGRTCNKTSKAIDGCELLCCGRGFHTAQVELAERCSCKFHWCCFVKCRQCQRLVELHTCR

SEQ ID NO: 11 - DNA, 1101 bp, Mus musculus
cgggagccttgcggccgctgccccgggctgggcgcgcacggcaccatgagcccccgttcgtgcctgcggtccctgcgactcctcgtcttcgccgtgttctcggccgccgcgagcaattggctgtacctggccaagctgtcatcggtgggcagcatctccgaagaggagacgtgcgagaaactcaaaggcctgatccagaggcaggtgcagatgtgcaaacggaaccttgaggtgatggactcagtgcgccgtggtgcccagctggccatcgaggagtgccaataccagttccggaaccggcgctggaactgttccacactggactccctccctgtctttgggaaggtggtgacacaagggacccgggaggcggcctttgtatacgccatctcttcagcaggtgtggcctttgcagtgacaagggcatgcagcagtggagaactggagaagtgtggctgtgaccggacagtgcacggggtcagcccacagggcttccagtggtcaggatgctcggacaacatcgcctatggcgtagccttctcacagtcctttgtggacgtccgggagaggagcaagggggcctcctccagccgggcactcatgaatcttcacaacaacgaggctggcaggaaggccatcttgacacacatgcgggtggagtgcaagtgtcacggggtgtcgggctcctgcgaggtaaagacgtgctggcgtgctgtaccgcccttccgccaggttggccacgcgctaaaggagaagtttgacggtgccacggaggtggagccacgacgcgtaggctcctcccgggcgctggtgcctcggaatgcacagttcaagccacatacagatgaggacctggtatacctggagcctagcccggacttctgtgagcaggacatccgcagtggcgtgctaggcacgaggggccgcacgtgcaacaagacatctaaagccattgacggctgcgagctactgtgctgtggccgcggcttccacacagcgcaagtggagctggccgagcgctgtggctgcaggttccactggtgctgcttcgtcaagtgccggcagtgccagcggctcgtggagatgcacacgtgccggtga

SEQ ID NO: 12 - protein, 351 aa, Mus musculus
MSPRSCLRSLRLLVFAVFSAAASNWLYLAKLSSVGSISEEETCEKLKGLIQRQVQMCKRNLEVMDSVRRGAQLAIEECQYQFRNRRWNCSTLDSLPVFGKVVTQGTREAAFVYAISSAGVAFAVTRACSSGELEKCGCDRTVHGVSPQGFQWSGCSDNIAYGVAFSQSFVDVRERSKGASSSRALMNLHNNEAGRKAILTHMRVECKCHGVSGSCEVKTCWRAVPPFRQVGHALKEKFDGATEVEPRRVGSSRALVPRNAQFKPHTDEDLVYLEPSPDFCEQDIRSGVLGTRGRTCNKTSKAIDGCELLCCGRGFHTAQVELAERCGCRFHWCCFVKCRQCQRLVEMHTCR

SEQ ID NO: 13 - DNA, 2082 bp, Homo sapiens
atgcgtggggcggcgcggctggggcggccgggccggagttgcctcccggggcccgcgctgagggccgccgccgcgcccgccctgctgcttgctcgttgcgctgttgccgctgctgccggcctgcgtgccgccgcccgtccgcggcccccggagctgcagtcggcttccgcggggcccagcgtgagtctctacctgagcgaggacgaggtgcgccggctgatcggtcttgatgcagaactttattatgtgagaaatgaccttattagtcactacgctctatcctttaatctgttagtacccagtgagacaaatttcctgcacttcacctggcatgcgaagtccaaggttgaatataagctgggattccaagtggacaatgttttggcaatggatatgccccaggtcaacatttctgttcagggggaagttccacgcactttatcagtgtttcgggtagagctttcctgtactggcaaagtagattctgaagttatgatactaatgcagctcaacttgacagtaaattcttcaaaaaattttaccgtcttaaattttaaacgaaggaaaatgtgctacaaaaaacttgaagaagtaaaaacttcagccttggacaaaaacactagcagaactatttatgatcctgtacatgcagctccaaccacttctacgcgtgtgttttatattagtgtaggggtttgttgtgcagtaatatttctcgtagcaataatattagctgttttgcaccttcataatatgaaaaggattgaactggatgacagcattagtgccagcagtagttcccaagggctgtctcagccatccacccagacgactcagtatctgagagcagacacgcccaacaatgcaactcctatcaccagttatcctaccttgcggatagagaagaacgacttgagaagtgtcactcttttggaggccaaaggcaaggtgaaggatatagcaatatccagagagaggataactctaaaagatgtactccaagaaggtacttttgggcgtattttccatgggattttaatagatgaaaaagatccaaataaagaaaaacaagcatttgtcaaaacagttaaagatcaagcttctgaaattcaggtgacaatgatgctcactgaaagttgtaagctgcgaggtcttcatcacagaaatcttcttcctattactcatgtgtgtatagaagaaggagaaaagcccatggtgatattgccttacatgaattgggggaatcttaaattgtttttacgacagtgcaagttagtagaggccaataatccacaggcaatttctcagcaagacctggtacacatggctattcagattgcctgtggaatgagctacctggccagaagggaagtcatccacaaagacctggctgccaggaactgtgtcattgatgacacacttcaagttaagatcacagacaatgccctctccagagacttgttccccatggactatcactgtctgggggacaatgaaaacaggccagttcgttggatggctcttgaaagtctggttaataacgagttctctagcgctagtgatgtgtgggcctttggagtgacgctgtgggaactcatgactctgggccagactccctacgtggacattgaccccttcgagatggccgcatacctgaaagatggttaccgaatagcccagccaatcaactgtcctgatgaattatttgctgtgatggcctgttgctgggccttagatccagaggagaggcccaagtttcagcagctggtacagtgcctaacagagtttcatgcagccctgggggcctacgtctgactcctctccaatcccacaccatcaggaagaaggtgcctgtcggggctcacttgaagcctgtcagggatgctttgtatctaacacaacgccaacagaagcacatttgtcttccagaacaccgtgccttagaaatggctttagaatctgaactttttaagacagacttaataatgtggcatattttctagatatcacttttattaggttgaactgaaagggtttttgtaaagcatttctaaggcacggtgttctggaagacaaatgtgc

SEQ ID NO: 14 - protein, 604 aa, Homo sapiens
MRGAARLGRPGRSCLPGPALRAAAAPALLLARCAVAAAAGLRAAARPRPPELQSASAGPSVSLYLSEDEVRRLIGLDAELYYVRNDLISHYALSFNLLVPSETNFLHFTWHAKSKVEYKLGFQVDNVLAMDMPQVNISVQGEVPRTLSVFRVELSCTGKVDSEVMILMQLNLTVNSSKNFTVLNFKRRKMCYKKLEEVKTSALDKNTSRTIYDPVHAAPTTSTRVFYISVGVCCAVIFLVAIILAVLHLHNMKRIELDDSISASSSSQGLSQPSTQTTQYLRADTPNNATPITSYPTLRIEKNDLRSVTLLEAKGKVKDIAISRERITLKDVLQEGTFGRIFHGILIDEKDPNKEKQAFVKTVKDQASEIQVTMMLTESCKLRGLHHRNLLPITHVCIEEGEKPMVILPYMNWGNLKLFLRQCKLVEANNPQAISQQDLVHMAIQIACGMSYLARREVIHKDLAARNCVIDDTLQVKITDNALSRDLFPMDYHCLGDNENRPVRWMALESLVNNEFSSASDVWAFGVTLWELMTLGQTPYVDIDPFEMAAYLKDGYRIAQPINCPDELFAVMACCWALDPEERPKFQQLVQCLTEFHAALGAYV

SEQ ID NO: 15 - DNA, 2463 bp, Mus musculus
agcggcgcgcgatgagctggccggccgcccccggctcggggctgtgaggcgctcggggccggggtgcgcggcggcgcggcgggcggcggacgctcctgctcggcggcggccatgcgcgcgggccggggcggcgtcccggggagcggcggcctgagggccccgccgccgccgctgctgctgctgctgctggcgatgctgcccgccgccgccccgcggtccccggccctggccgccgctcctgcgggacccagcgtgagcctctacctgagcgaggacgaggtgcgccggctgcttggtcttgatgcagagctttactatgtgagaaatgacctcatcagtcactacgctctgtcctttaacctgctagtgcccagtgagacaaacttcctgcacttcacttggcatgcaaagtccaaggttgaatataagctgggattccaagtagacaactttgtggctatgggcatgccccaggtcaatatttctgctcaaggggaggtcccacgcactttatcagtgtttcgggtcgagctttcttgtaccggcaaagtcgactctgaagtcatgattctaatgcagctcaatctgacagtgaattcctcaaaaaattttacagttttaaattttaaacgaaggaaaatgtgctacaaaaaacttgaagaagtaaaaacttcagccttggacaaaaacactagcagaactatttatgaccctgtccatgcagcgccaacgacttccacgcgtgtgttttacatcagtgtaggggtttgctgtgcagtgatatttcttgtagcaataatattagccgttttgcaccttcatagcatgaaaaggattgaactggatgacagcatcagcgccagcagtagttcccaggggctgtctcagccgtctacccagacgacccagtatctgagagctgacacacccaacaatgcaacgcctatcaccagctcctcaggttatcctaccttgcggatagagaagaacgacttgcgaagtgtcactcttctggaagccaaagccaaggtgaaggatatcgcaatatccagagaaaggatcacactgaaagatgtcctccaagaaggtacttttgggcgtattttccatgggattttagtagatgaaaaagatccaaataaagagaagcaaacatttgtaaaaacagttaaagaccaagcatctgaagttcaggtgacgatgatgctcaccgagagctgcaagcttcgaggtctgcaccacagaaacctccttcctattactcatgtgtgcatagaagaaggagaaaagcccatggtggtattgccatacatgaattgggggaatcttaaattatttcttcggcagtgcaaattagtagaagccaataatccacaggcaatttcccagcaagatctggtccatatggctattcagattgcctgcgggatgagctacctggcgaggagagaagtgatccatagagacctggctgctaggaactgtgtcatcgacgacactcttcaagtcaagatcacagacaatgccctttccagagacttgtttcctatggactaccactgcctaggggacaacgagaacaggccagtgagatggatggctctggaaagtctggttaataatgagttctctagtgctagtgacgtgtgggcctttggagtgacgctgtgggagctcatgactctgggccagacgccctacgtggacatcgacccctttgagatggccgcttacctgaaagatggttaccgaatagcccagccaatcaactgccctgatgaactgtttgctgtgatggcctgttgctgggccttggaccctgaggagaggcctaagttccagcagctggtccagtgcctcacagagttccacgctgccctgggagcctacgtctgacttctctcccagctccgccactcagaagaaagtgcctgtctgtcacggatgcccctcgtgcagcgcagtgcctgcaggggcacactgtctccagatcacccagccttagcagtgcttccaaacctcagcttttaacgatgacgtaataacgcagagtgttttctagataccacttttactaggttgaaccaaaaggggttttgtacattttttggccaaaaatttttttaaaaatatagtgactttggactagggggtgcattcttttaaaaaatgaataaacagttttaaaaattatttagacacagatatttggaatagctgtcttagtgccaactgcttttattcttaatgttttacttcattgaagtaatgtaggtgactcacctttaaagctattttaatattttggtcactacttttgggagtggtttgtcttcaaaatgcggtacttttcttaggaaaaacaacattacgaagttgtctggtttgccttatacgaagaaagatagtatattaggaaaattgaaaaagtaaaaagaaaaagaaattacaaagtaaaaaaaa

SEQ ID NO: 16 - protein, 594 aa, Mus musculus
MRAGRGGVPGSGGLRAPPPPLLLLLLAMLPAAAPRSPALAAAPAGPSVSLYLSEDEVRRLLGLDAELYYVRNDLISHYALSFNLLVPSETNFLHFTWHAKSKVEYKLGFQVDNFVAMGMPQVNISAQGEVPRTLSVFRVELSCTGKVDSEVMILMQLNLTVNSSKNFTVLNFKRRKMCYKKLEEVKTSALDKNTSRTIYDPVHAAPTTSTRVFYISVGVCCAVIFLVAIILAVLHLHSMKRIELDDSISASSSSQGLSQPSTQTTQYLRADTPNNATPITSSSGYPTLRIEKNDLRSVTLLEAKAKVKDIAISRERITLKDVLQEGTFGRIFHGILVDEKDPNKEKQTFVKTVKDQASEVQVTMMLTESCKLRGLHHRNLLPITHVCIEEGEKPMVVLPYMNWGNLKLFLRQCKLVEANNPQAISQQDLVHMAIQIACGMSYLARREVIHRDLAARNCVIDDTLQVKITDNALSRDLFPMDYHCLGDNENRPVRWMALESLVNNEFSSASDVWAFGVTLWELMTLGQTPYVDIDPFEMAAYLKDGYRIAQPINCPDELFAVMACCWALDPEERPKFQQLVQCLTEFHAALGAYV

SEQ ID NO: 17 - DNA, 63 bp, Artificial sequence
  SiRNAi-1 targets human Ryk
gatccccgtccaggttgaatataagttcaagagacttatattcaaccttggactttttggaaa

SEQ ID NO: 18 - DNA, 64 bp, Artificial sequence
  SiRNAi-1 targets human Ryk
agcttttccaaaaagtccaaggttgaatataagtctcttgaacttatattcaaccttggacggg

SEQ ID NO: 19 - DNA, 64 bp, Artificial sequence
  SiRNA-2 targets human Ryk
gatccccgatggttaccgaatagcccttcaagagagggctattcggtaaccatctttttggaaa

SEQ ID NO: 20 - DNA, 64 bp, Artificial sequence
  SiRNA-2 targets human Ryk
agcttttccaaaaagatggttaccgaatagccctctcttgaagggctattcggtaaccatcggg

SEQ ID NO: 21 - DNA, 64 bp, Artificial sequence
  siRNA oligos targetting Dishevlled-2
gatcccccatggagaagtacaacttcttcaagagagaagttgtacttctccatgtttttggaaa

SEQ ID NO: 22 - DNA, 64 bp, Artificial sequence
  siRNA oligos targetting Dishevlled-2
agcttttccaaaaacatggagaagtacaacttctctcttgaagaagttgtacttctccatgggg

SEQ ID NO: 23 - DNA, 63 bp, Artificial sequence
  siRNA oligos targeting Dishevelled-3
gatccccgttcttcttcaagtctatgttcaagagacatagacttgaagaagaactttttggaa

SEQ ID NO: 24 - DNA, 64 bp, Artificial sequence
  siRNA oligos targeting Dishevelled-3
agcttttccaaaaagttcttcttcaagtctatgtctcttgaacatagacttgaagaagaacggg

SEQ ID NO: 25 - DNA, 23 bp, Artificial sequence
  Amplification primer
aggtgacaatgatgctcactgaa

SEQ ID NO: 26 - DNA, 21 bp, Artificial sequence
  Amplification primer
tgtgatgaagacctcgcagct

SEQ ID NO: 27 - DNA, 23 bp, Artificial sequence
  Amplification primer
caggtgacaatgatgctcactga

SEQ ID NO: 28 - DNA, 22 bp, Artificial sequence
  Amplification primer
gtgatgaagacctcgcagctta

SEQ ID NO: 29 - DNA, 23 bp, Artificial sequence
  Amplification primer
ggtggtctcctctgacttcaaca

SEQ ID NO: 30 - DNA, 20 bp, Artificial sequence
  Amplification primer
gcgtcaaaggtggaggagtg